(19) United States
(12) Patent Application Publication
(10) Pub. No.: US 2024/0398969 A1
(43) Pub. Date: Dec. 5, 2024

(54) ANTIBODY-DRUG CONJUGATES FOR TREATING INFECTIONS OF HUMAN IMMUNODEFICIENCY VIRUS (HIV)

(71) Applicant: TaiMed Biologics Inc., Taipei City (TW)

(72) Inventors: Chu-Ling WEN, Taipei City (TW); Yu-Hong MAO, Taipei City (TW); Chin-Ming CHANG, Taipei City (TW)

(21) Appl. No.: 18/679,860

(22) Filed: May 31, 2024

Related U.S. Application Data
(60) Provisional application No. 63/505,573, filed on Jun. 1, 2023.

(51) Int. Cl. A61K 47/68

(52) U.S. Cl. CPC A61K 47/6849; A61K 47/6803; A61K 47/6889

(57) ABSTRACT

The present invention provides an antibody-drug conjugate (ADC) for treating HIV infection, comprising an antibody that binds to CD4, conjugated via a linker to a small-molecule drug capable of treating or preventing HIV infection.

### CROSS REFERENCE

This Non-provisional application claims the priority under 35 U.S.C. § 119 (a) on U.S. Patent Provisional Application No. 63/505,573 filed on Jun. 1, 2023, the entire contents of which are hereby incorporated by reference.

### FIELD OF THE INVENTION

The present invention relates to a new approach for treating an infection of human immunodeficiency virus (HIV). In particular, the present invention relates to a new conjugate of an antibody and a small-molecule drug (ADC) for treating HIV infection.

### BACKGROUND OF THE INVENTION

Acquired Immunodeficiency Syndrome (AIDS) is an infectious disease caused by human immunodeficiency virus (HIV). Recently, the HIV epidemic has become more and more serious.

It is known that HIV-1 entry is triggered by interaction of the viral envelope (Env) glycoprotein gp120 with domain 1 (D1) of the T-cell receptor CD4. Binding of CD4 by gp120 induces extensive conformational changes in gp120 leading to formation and exposure of a structure called the co-receptor (coR) binding site, also known as the CD4-induced (CD4i) epitope, in the gp120 protein. (Moore et al., Proc. Natl. Acad. Sci. USA 100 (10): 10598-602, 2003.)

It was also reported that Latent HIV-1 persists in resting memory CD4 T cells, even in patients receiving highly active antiretroviral therapy (HAART). Even in treated patients who have had no detectable viremia for as long as 7 years, the reservoir decays so slowly (t_{1/2}=44 months) that eradication is unlikely. (Sillciano, J. D. et al., Nature Med. 9:727-28, 2003.) These latently infected cells do not express viral proteins and hence remain invisible to the immune system. Latency is associated with transcriptional silencing of the integrated provirus and driven. If activated, however, they can ignite new rounds of viral replication—a risk that forces patients to remain on therapy indefinitely. Current treatments for HIV infection limit replication of the virus but not eradicate it, as the viral genome remains integrated into the DNA of memory CD4⁺ T cells. It has been reported that the potential for a new therapeutic approach (“shock and kill”) that involves activating viral replication was tested. (Archin, N. M. et al., AIDS Res. Hum. Retroviruses 25:207-212, 2009.) Theoretically, drugs that reverses latency might lead sequentially to HIV RNA synthesis, viral protein production, release of HIV particles and killing of the infected cell by the virus or by the patient's immune system. Therefore, a cure might be possible if the latent virus in all infected cells can be forced out of its hiding place, leading ultimately to the death of the cells and to the elimination of the viral reservoir. Such a potential approach is known as “shock and kill,” see FIG. 1. (Deeks, S., Nature 487 (7408): 439-740, 2012.) However, there are some concerns raised by this potential therapeutic approach, including for example, the drugs like an HDAC inhibitor are toxic; a long-term treatment of HIV infection is required; such anti-latency drugs require a costly and inconvenient leukapheresis before and after drug exposure.

It is still desirable to develop a new approach to treat HIV infection.

### BRIEF SUMMARY OF THE INVENTION

Accordingly, the present invention provides a new approach for treating HIV infection, wherein an antibody-drug conjugate (ADC) is dedicated for the cocktail-combination medication in a single formation by combining and incorporating a small-molecule drug as an integrated compartment into an antibody, which is an all-in-one molecule as a combination medicament against HIV infection.

In one aspect, the present invention provides an antibody-drug conjugate (ADC) for treating HIV infection comprising an antibody that binds to CD4 (anti-CD4), conjugated via a linker to one or more small-molecule drug capable of treating or preventing HIV infection.

According to the present invention, a new approach for treating HIV infection is provided to activate HIV expression from latent reservoirs and lead to virus elimination and ultimately cure of the HIV infection with a conjugate of an antibody and a small-molecule drug.

On the other hand, the present invention also provides an approach for treating HIV infection with a single molecule dedicated for a cocktail-combination medication against HIV.

In one embodiment of the invention, the antibody is an anti-CD4 antibody, a binding protein, peptide or fragment capable of binding to CD4, or a variant, derivative or modified form thereof.

In one embodiment of the invention, the antibody is an immediate-release anti-CD4 or a sustained-release anti-CD4.

In one example of the invention, the immediate-release anti-CD4 is TMB-355 or a variant, derivative or modified form thereof, referring to a non-long-acting (non-LA) antibody that binds to CD4.

In another example of the invention, the glycan-modified anti-CD4 is TMB-360 or a variant, derivative or modified form thereof, referring to a monoclonal antibody that binds to CD4.

In another example of the invention, the sustained-release anti-CD4 is TMB-365 or a variant, derivative or modified form thereof, referring to a long-acting (LA) antibody that binds to CD4.

In another example of the invention, the bispecific anti-CD4 is TMB-370 or a variant, derivative or modified form thereof, referring to a fusion antibody that binds to CD4.

In one embodiment of the invention, the small-molecule drug is a functional cure-oriented latency reversing agent (LRA) or a treatment-driven antiretroviral agent (ARV).

In one example of the invention, the LRA is a drug capable to drive HIV out of hiding in the latently infected cells, such as an HDAC inhibitor.

In another example of the invention, the ARV is an antiretroviral agent, such as an integrase strand transfer inhibitor (INSTI), a reverse transcriptase inhibitor (RTI) (including a nucleoside reverse transcriptase inhibitor (NRTI), a non-nucleoside reverse transcriptase inhibitor (NNRTI), and nucleoside reverse transcriptase translocation inhibitor (NRTTI)), a protease inhibitor (PI), a capsid assembly inhibitor (CAI), an entry inhibitor (EI), an attachment inhibitor or a maturation inhibitor.

In the present invention, one particular example of the conjugate ADC is a conjugate of anti-CD4, such as TMB-355, TMB-360, TMB-365, or TMB-370, and a functional cure-oriented latency reversing agent (LRA), such as an HDAC inhibitor.

In the present invention, another particular example of the conjugate ADC is a conjugate of anti-CD4, such as TMB-355, TMB-360, TMB-365, or TMB-370, and a treatment-driven antiretroviral agent (ARV), such as an integrase strand transfer inhibitor (INSTI), a reverse transcriptase inhibitor (RTI) including a nucleoside reverse transcriptase inhibitor (NRTI) and a non-nucleoside reverse transcriptase inhibitor (NNRTI), and nucleoside reverse transcriptase translocation inhibitor (NRTTI)), a protease inhibitor (PI), a capsid assembly inhibitor (CAI), an entry inhibitor (EI), an attachment inhibitor or a maturation inhibitor.

In another aspect, the present invention provides a pharmaceutical composition for treating HIV infection comprising a therapeutically effective amount of the conjugate ADC according to the invention, and a pharmaceutically acceptable carrier.

In a further aspect, the present invention provides a method for treating HIV infection in a subject, comprising administering to the subject the conjugate ADC according to the invention in a therapeutically effective amount or the composition thereof.

In a yet aspect, the present invention provides a method for treating HIV infection in a subject, comprising administering to the subject the conjugate ADC according to the invention or the composition thereof, in combination with one or more other therapeutical agents for the treatment of HIV infection.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The drawings presenting the preferred embodiments of the present invention are aimed at explaining the present invention. It should be understood that the present invention is not limited to the preferred embodiments shown.

FIG. 1 provides NMR of SMCC-Romidepsin-Spy (4) and (5).

FIG. 2 provides RP-HPLC of SMCC-Romidepsin-SPy (4) and (5).

FIG. 3 provides RP-HPLC of TMB355-Biotin (6).

FIG. 4 provides RP-HPLC of TMB355-MCC-Romidepsin-Spy (7) and (8).

FIG. 5 provides images of intact mass of TMB355-MCC-Romidepsin-Spy (7) and (8).

FIG. 6 provides images of reduced mass of TMB355-MCC-Romidepsin-Spy (7) and (8).

FIG. 7 provides images of Gel Electrophoresis of TMB355-MCC-Romidepsin-Spy (7) and (8).

FIG. 8 provides the results of the analysis on the CD4 binding of TMB-355 and TMB355-SMCC-Romidepsin-Spy 6 and 7.

FIG. 9 provides the enrichment of CD4+ Jurkat T cells.

FIG. 10 provides the internalization of TMB-355-SMCC-Romindepsin-Spy (7) and (8).

FIG. 11 provides the results of the deconvolution microscopy-based internalization assay of TMB-355-SMCC-Romindepsin-Spy (7) and (8)

### DETAILED DESCRIPTION OF THE INVENTION

Unless otherwise defined herein, scientific and technical terms used herein have the meaning that is commonly understood by those of ordinary skill in the art.

As used herein, the singular forms “a”, “an”, and “the” include plural referents unless the context clearly dictates otherwise. Thus, for example, reference to “a sample” includes a plurality of such samples and equivalents thereto known to those skilled in the art.

The term “antibody” is used herein broadly and encompasses an intact antibody molecule, including intact polyclonal, monoclonal, monospecific, polyspecific, chimeric, humanized, human, primatized, single-chain, single-domain, synthetic and recombinant antibodies, and antibody fragments that have a desired activity or function. The term “antibody” also includes fragments or an intact antibody, or “antibody fragments”, including particularly antigen-binding fragments, of an intact antibody. Examples of the antigen-binding fragments include but are not limited to Fab fragments (consisting of the VL, VH, VL and CH1 domains), Fab′ fragments (which differs from Fab fragments by having an additional few residues at the C-terminus of the CH1 domain including one or more cysteines from the antibody hinge region), (Fab′)₂ fragments (formed by two Fab′ fragments linked by a disulphide bridge at the hinge region), Fd fragments (consisting of the VH and CH1 domains), Fv fragments (referring to a dimer or one heavy and one light chain variable domain in tight, non-covalent association which contains a complete antigen recognition and binding site), dAb fragment (consisting of a VH domain), single domain fragments (VH domain, VL domain VHH domain, VNAR domain), isolated CDR regions, scFv (or “single chain Fv”, referring to a fusion of the VL and VH domains, linked together via a linker), and other antibody fragments that retain antigen-binding function.

The term “fragment” as used herein refers to a physically contiguous portion of the primary structure of a biomolecule. In case of proteins, a fragment may be defined by a contiguous portion of the amino acid sequence of a protein and may be at least 3-5 amino acids, at least 6-10 amino acids, at least 11-15 amino acids, at least 16-24 amino acids at least 25-30 amino acids, at least 30-45 amino acids and up to the full length of the protein minus a few amin acids.

The term “subject” as used herein refers to any humans and non-human mammals, such as primates, rodents, monkeys, dogs, cats and so on.

The present invention provides a conjugate (“ADC”) for treating HIV infection, which comprises an antibody that binds to CD4 (“anti-CD4”), conjugated via a linker to a small-molecule drug capable of treating or preventing HIV infection.

As used herein, the term “anti-CD4”, “anti-CD4 antibody” or “anti-CD4 antibodies” refers to any antibody variant, derivative, or modified form thereof, which has an antigen-binding site that binds to an epitope on the CD4 receptor. The anti-CD4 antibodies have been described in the art and can also readily generated as the protein sequence of the CD4 receipt is available to those skilled in the art. A specific example of an anti-CD4 antibody is TMB-355, known as Ibalizumab, TNX-355 or hu5A8, which is a humanized, anti-CD-4 monoclonal antibody, and potently blocks infection by a broad spectrum of HIV-1 isolates. The anti-CD4 antibody may be any derivatives of TMB-355, such as a bispecific anti-CD4 antibody as disclosed in U.S. Pat. No. 8,637,024, and a glycan-modified forms as disclosed in U.S. Pat. Nos. 9,7902,276 B2, and 9,587,022 B2 respectively, an anti-CD4 antibody that has an increased half-life in vivo as compared to TMB-355 as disclosed in US Patent Publication Nos. 2013/0195881 A1, and a histidine-mutated anti-HIV antibody disclosed in US Patent No. 2021/0054054 A1 all of which hereby incorporated by reference in their entirety.

In some examples of the present invention, the suitable anti-CD4 antibodies are listed in Table 1 below.

TABLE 1
A list of suitable anti-CD4 antibodies

| Antibody | Antigen + [Antibody Modifications] | U.S. Pat. No. |
|---|---|---|
| ibalizumab | 1. CD4 | U.S. Pat. No. 5,750,105A |
| (TMB-355; | 2. (IgG4, G4) ibalizumab; TMB-355 | U.S. Pat. No. 5,756,096A |
| IBA) - IgG1- | (TNX-355), G4H (TMB-358), G4D, | U.S. Pat. No. 5,871,732A |
| backbond, | 3. (IgG1, G1) ibalizumab; MV1 (TMB-359) | U.S. Pat. No. 6,136,310A |
| ibalizumab | MV1 is the backbone/scaffold of TMB-360 | U.S. Pat. No. 7,041,293B1 |
| derivative | and TMB-365; that is, TMB-360 and | U.S. Pat. No. 7,338,658B2 |
| iMAb (MV1) - | TMB-365 were derived from MV1 (TMB-359). | U.S. Pat. No. 7,452,534B1 |
| IgG1- | US Patent Application US20130195881A1 | U.S. Pat. No. 7,923,221B1 |
| backbone | described how G4H, G4D and MV1 | US20100021460A1 |
| | were genetically engineered. | US20130195881A1 |
| bispecific | CD4 × ENV* (gp120) | US20120121597A1 |
| antibody | CD4 × ENV* (gp41) | U.S. Pat. No. 8,637,024B2 |
| (bispecific | | U.S. Pat. No. 9,587,012B2 |
| fusion | | U.S. Pat. No. 9,884,905B2 |
| protein) | | |
| (TMB-370) | | |
| TMB-360 | CD4 | U.S. Pat. No. 9,587,022B2 |
| | Glycan modification | U.S. Pat. No. 9,790,276B2 |
| TMB-365 | CD4 | U.S. Pat. No. 9,587,022B2 |
| | Glycan modification | U.S. Pat. No. 9,790,276B2 |
| | Histidine modification | US20210054054A1 |

*ENV: HIV envelop glycoproteins (gp120, gp41)

According to the invention, the small-molecule drug may be any drug for treating or preventing HIV infection, including but not limited to an HDAC inhibitor, which may be one selected from the group consisting of vorinostat, romidepsin, chidamide, panobinostat, and belinostat.

In some examples of the present invention, the suitable small-molecule drugs are listed in Table 2.

TABLE 2
A list of suitable small-molecule drugs

| Mechanism of Action | Drug | Route |
|---|---|---|
| nucleoside/nucleotide | Abacavir | Oral |
| reverse transcriptase | Emtricitabine | Oral |
| inhibitor (NRTI) | Lamivudine | Oral |
| | Tenofovir disoproxil fumarate (TDF) | Oral |
| | Tenofovir Alafenamide (TAF) | Oral |
| | Zidovudine | Oral |
| Nucleoside reverse | Islatravir | Oral |
| transcriptase translocation | | |
| inhibitor (NRTTI) | | |
| Non-nucleoside reverse | Doravirine | Oral |
| Transcriptase inhibitor | Efavirenz | Oral |
| (NNRTI) | Etravirine | Oral |
| | Nevirapine | Oral |
| | Dapivirine | Oral |
| | Rilpivirine | Oral |
| | | IM |
| protease inhibitor (ADC | Atazanavir | Oral |
| using PK-booster-free | Darunavir | Oral |
| protease inhibitor) (PI) | Fosamprenavir | Oral |
| | Tipranavir | Oral |
| Integrase strand transfer | Cabotegravir | IM |
| inhibitor (INSTI) | | Oral |
| | Dolutegravir | Oral |
| | Elvitegravir | Oral |
| | Bictegravir | Oral |
| | Raltegravir | Oral |
| Capsid inhibitor (CAI) | Lenacapavir | SC |
| | | Oral |

The term “a link” as used herein refers to a spacer for linking the anti-CD4 and the small-molecule drug, which may be cleavable and/or non-cleavable linker, such as a protease or a lysosome. The linker may also be a chemical moiety, a fragment of amino acids, a saturated or unsaturated carbon chain that connects the anti-CD4 to the small-molecule antibody.

In the invention, the linker is selected from the group consisting of:
maleimidocaproyl (mc),
maleimidocaproyl-p-aminobenzylcarbamate,
maleimidocaproyl-valine-citrulline-para-aminobenzylcarbamoyl (val-cit),
maleimidocaproyl-peptide-aminobenzylcarbamate linkers,
N-succinimidyl 3-(2-pyridyldithio) proprionate (SPP),
4-succinimidyl-oxycarbonyl-2-methyl-2-(2-pyridyldithio)-toluene (SMPT),
N-succinimidyl 3-(2-pyridyldithio) propionate (SPDP),
N-succinimidyl 4-(2-pyridyldithio) butyrate (SPDB),
2-iminothiolane,
Sacetylsuccinic anhydride,
disulfide benzyl carbamate,
carbonate,
hydrazone linkers,
N-(a-Maleimidoacetoxy) succinimide ester,
N-[4-(p-Azidosalicylamido)butyl]-3′-(2′-pyridyldithio) propionamide (AMAS),
N-[b-Maleimidopropyloxy]succinimide ester (BMPS),
[N-e-Maleimidocaproyloxy]succinimide ester (EMCS),
N-[g-Maleimidobutyryloxy]succinimide ester (GMBS),
Succinimidyl-4-[NMaleimidomethyl]cyclohexane-1-carboxy-[6-amidocaproate] (LC-SMCC),
Succinimidyl 6-(3-[2-pyridyldithio]-propionamido) hexanoate (LC-SPDP),
m-Maleimidobenzoyl-N-hydroxysuccinimide ester (MBS),
N-Succinimidyl [4-26 iodoacetyl]aminobenzoate (SIAB),
Succinimidyl 4-[Nmaleimidomethyl]cyclohexane-1-carboxylate (SMCC),
N-Succinimidyl 3-[2-pyridyldithio]-propionamido (SPDP),
[N-e-Maleimidocaproyloxy]sulfosuccinimide ester (Sulfo-EMCS),
N-[g-Maleimidobutyryloxy]sulfosuccinimide ester (Sulfo-GMBS),
4-Sulfosuccinimidyl-6-methyl-α-(2-pyridyldithio) toluamido]hexanoate) (Sulfo-LC-SMPT),
Sulfosuccinimidyl 6-(3′-[2-pyridyldithio]-propionamido) hexanoate (Sulfo-LC-SPDP),
m-Maleimidobenzoyl-Nhydroxysulfosuccinimide ester (Sulfo-MBS),
N-Sulfosuccinimidyl [4-iodoacetyl]aminobenzoate (Sulfo-SIAB),
Sulfosuccinimidyl 4-[Nmaleimidomethyl]cyclohexane-1-carboxylate (Sulfo-SMCC),
Sulfosuccinimidyl 4-[pmaleimidophenyl]butyrate (Sulfo-SMPB),
ethylene glycol-bis(succinic acid Nhydroxysuccinimide ester) (EGS),
disuccinimidyl tartrate (DST),
1,4,7,10-tetraazacyclododecane-1,4,7,10-tetraacetic acid (DOTA),
diethylenetriaminepentaacetic acid (DTPA), and
thiourea linkers.

According to the present invention, the example of the linker (presented by the letter “L” in the compound formulae) is one selected from the group consisting of

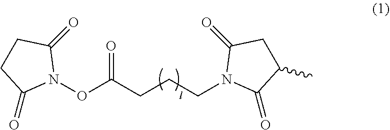

wherein 1 is an integer from 1 to 6;

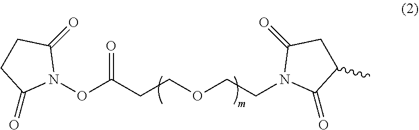

wherein m is an integer from 1 to 6;

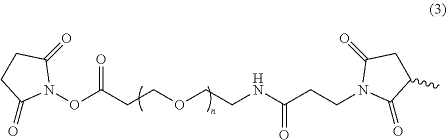

wherein n is an integer from 1 to 6;

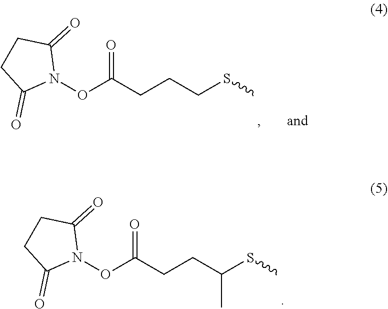

In one example, the preparation of the conjugate ADC is listed and illustrated in the following schemes, wherein L represents a linker.

### Scheme 1 Showing a Synthetic Route to Compound (A)

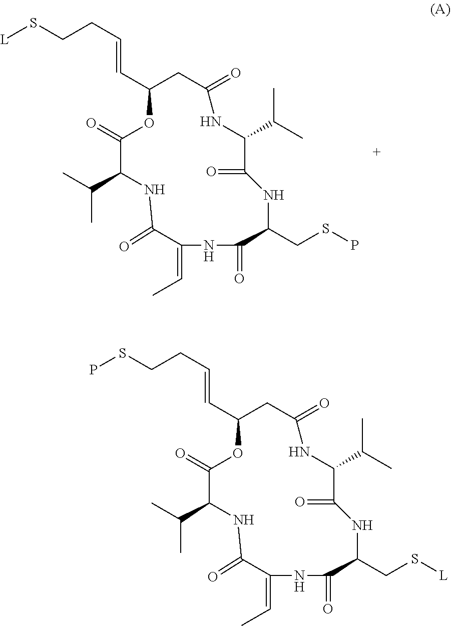

Commercially available romidepsin is reduced to generate dithio groups, such as 1,4-Dithiothreitol in the aqueous methanol. The resulting dithio derivative can react with a proper linker such as SMCC and then thio protection agents such as cysteine or dipyridinyldisulfane in an inert solvent such as THF to give the desired Linker-payload compound (A). The scheme 1 is illustrated as follows:

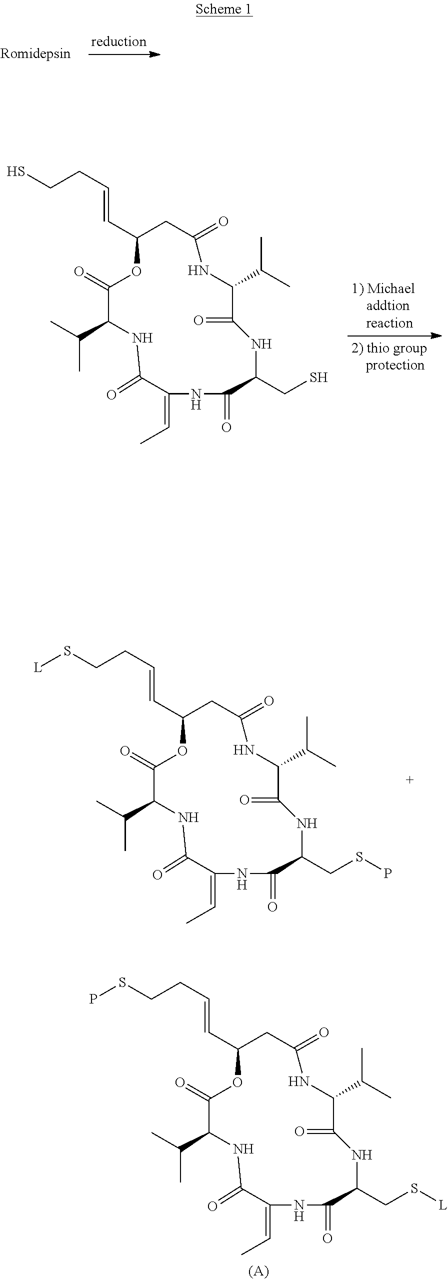

In the structures, P represents a protecting group, having a structure of

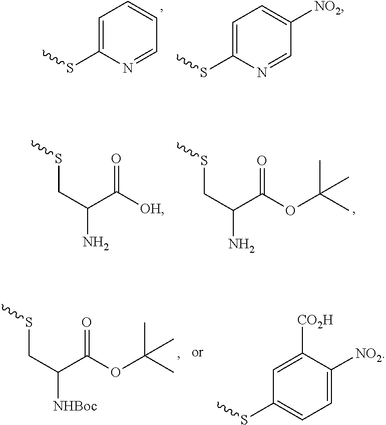

### Scheme 2 Showing a Synthetic Route to Compound (B)

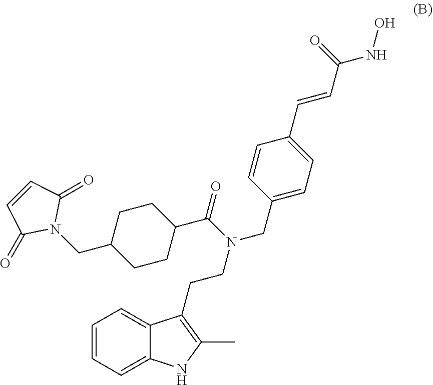 [SMILES: Cc1[nH]c2ccccc2c1CCN(Cc1ccc(/C=C/C(=O)NO)cc1)C(=O)C1CCC(CN2C(=O)C=CC2=O)CC1]

Compound (B) can be synthesized by reacting between panobinostat and SMCC in the presence of DBU to give the product (B) (see Scheme 2). An alternative to synthesize the series of compounds with variety of linkers to compound (B) can be achieved by replacing SMCC linker to the compounds list in L to produce linker-panobinostat analogs.

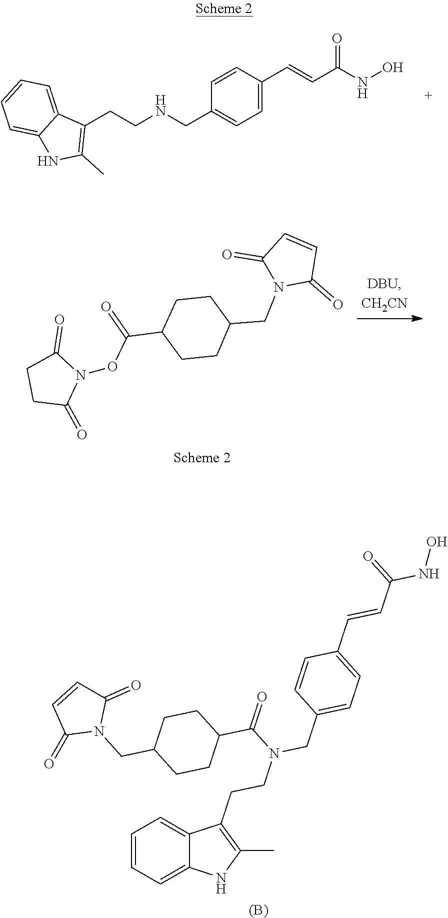

### Scheme 3 Showing a Synthetic Route to Compound (C)

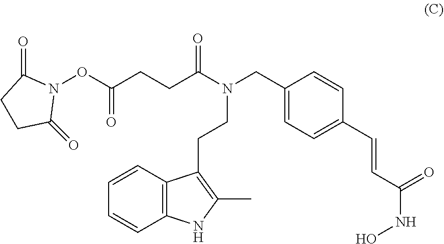 [SMILES: Cc1[nH]c2ccccc2c1CCN(Cc1ccc(/C=C/C(=O)NO)cc1)C(=O)CCC(=O)ON1C(=O)CCC1=O]

Many commercially available or can be prepared by the literature methods di-carboxylic acid analogs can be used as a linker such as succinic acid anhydride, hexanedioic acid, maleic anhydride, and fumaric acid, but not limit to linear saturated, branched-chain and unsaturated dicarboxylic acid derivatives. Coupling of panobinostat and di-carboxylic acid analogs can be prepared by conventional methods familiar to those skilled in the art. When the activating carboxylation groups are desired such as compound (C), the carboxylic acid intermediate can be converted to target final compounds by standard coupling reaction condition in an inert solvent, such as dichloromethane with N-hydroxysuccinimide in the presence of coupling reagent. One exemplary preparation of compound (C) is shown in Scheme 3.

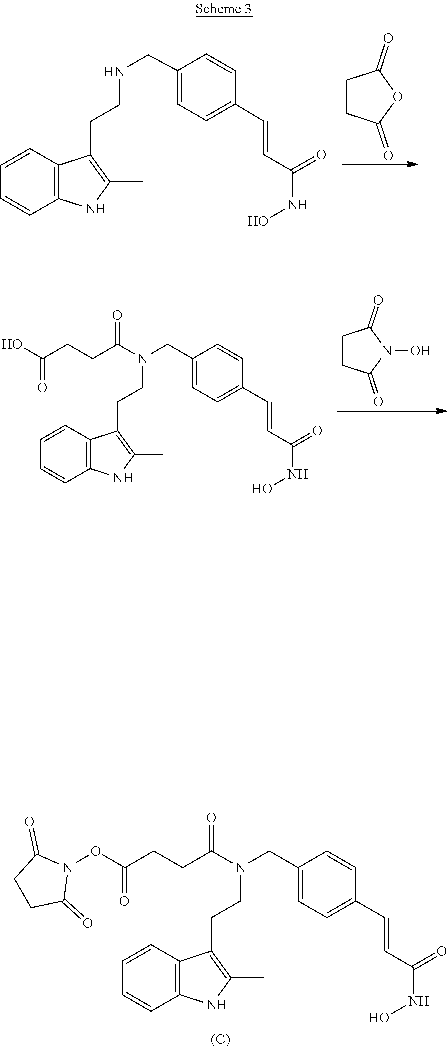

### Scheme 4 Showing a Synthetic Route to Compound (D)

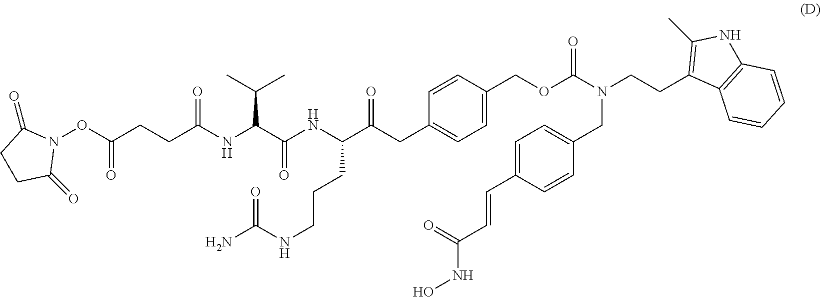

A protease cleavable linker-payload compound can be designed and prepared in the linker-panobinostat series. The preparation of compound (D) is shown in Scheme 4. Protease cleavable linker, such as commercially available Val-Cit dipeptide linker (E), can react with panobinostat in the presence of organic base such as DIEPA in a proper solvent to give compound (F). Separation of unwanted side products and purification of intermediate may be achieved by chromatography on silica gel. The BOC protection of compound (F) can be removed by conventional methods familiar to those skilled in the art. The free amine analog (G) can react with succinic acid anhydride and then convert the resulting carboxylic acid to activating group with N-hydroxysuccinimide in the presence of coupling reagent in an inert solvent.

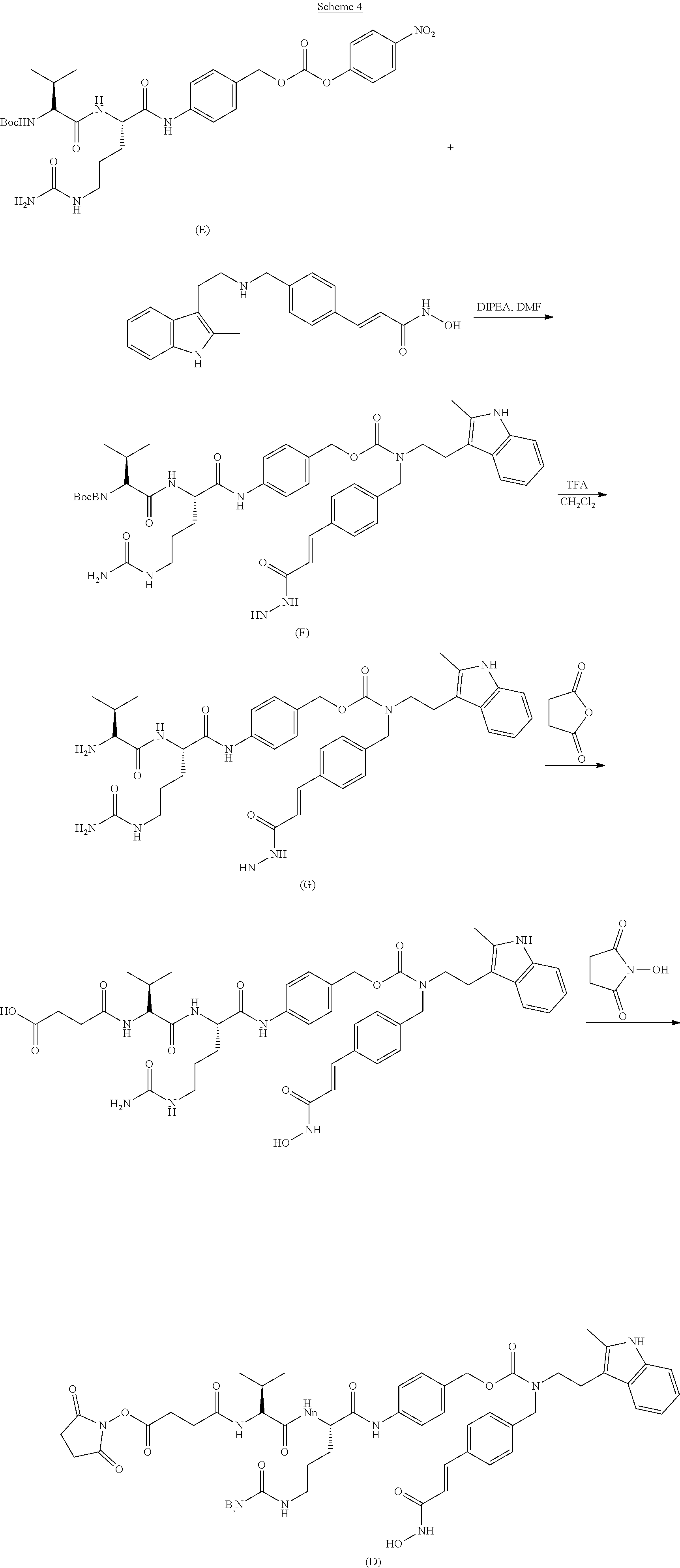

### Scheme 5 Showing a Synthetic Route to Compound (D)

Linkage between anti-CD4 antibody, such as TMB-355 (known as Ibalisumab), and linker-payload compounds, may provide the designed antibody drug conjugate (ADC) by the literature methods. In general, antibody can provide free cysteines or lysines to connect to linker-payload compounds by Michael type addition reaction or amide bond formation reaction. In Scheme 5, free cysteines of ibalizumab can react with maleimide of compound (B) by the methods familiar to those skilled in the art to offer the final ADC product (H).

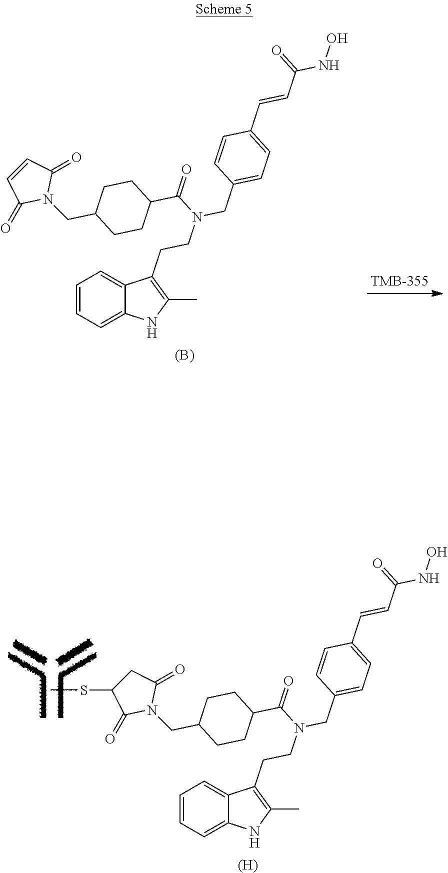

### Scheme 6 Showing the Reaction to Give the ADC Products

Alternatively, TMB-355 (known as Ibalizumab) can also provide the free lysine group to form the amide bond to connect between ibalizumab and linker-payload compounds (A), (C), and (D). The reaction is illustrated by using known methods as shown in Scheme 6 to give the desired ADC products (M), (J) and (K).

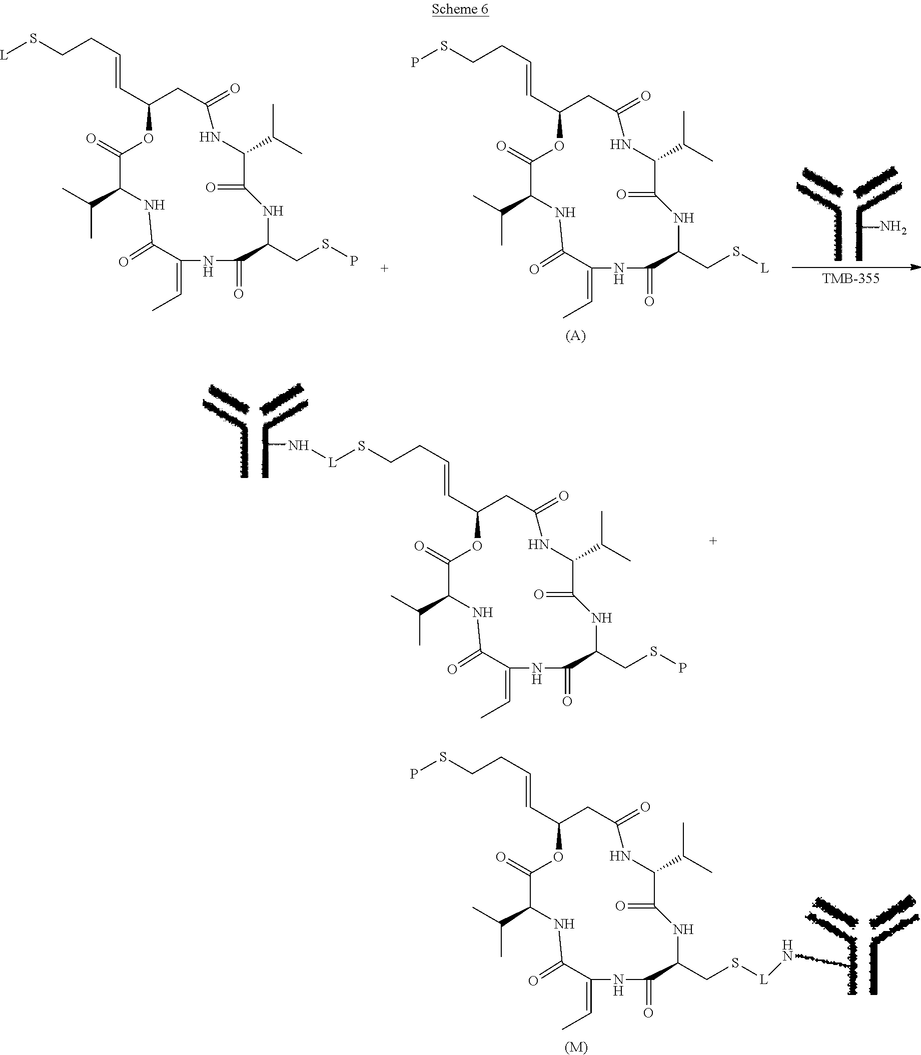

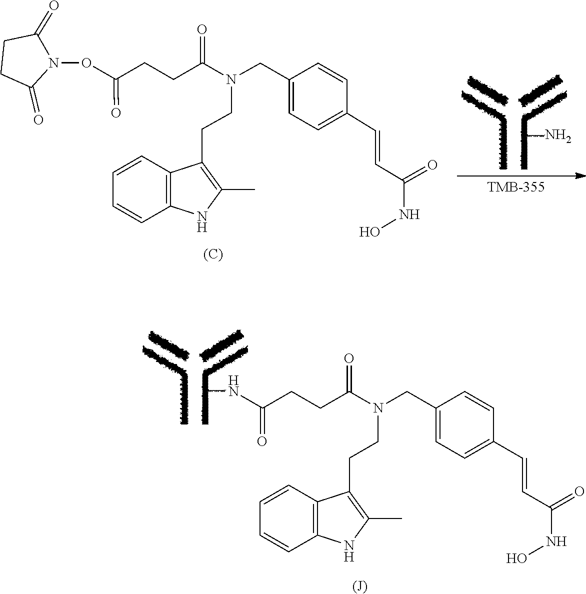

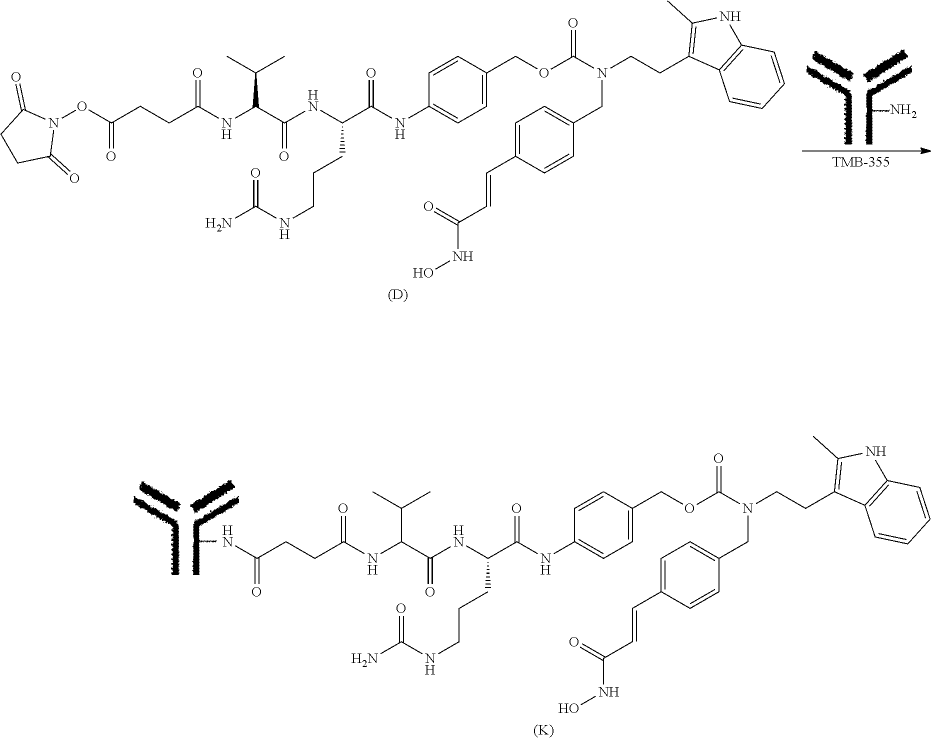

### Example: Preparation of SMCC-Romidepsin-Spy

This example provides a process for preparing a conjugate ADC according to the present invention, wherein the conjugate is composed of TMB-355 (a CD4⁺ mAb) and Romidepsin (a HDAC inhibitor). The brief scheme is given below.

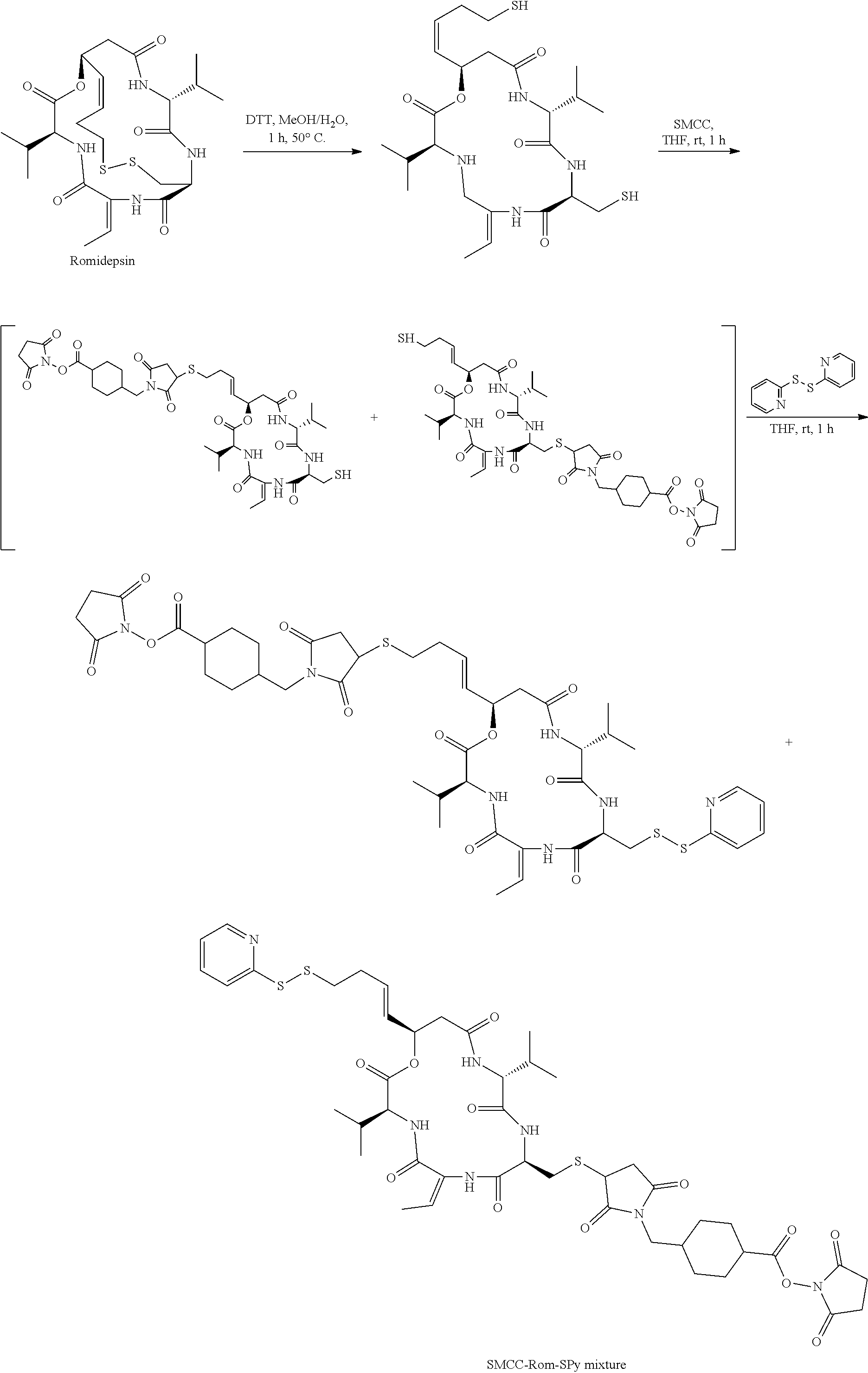

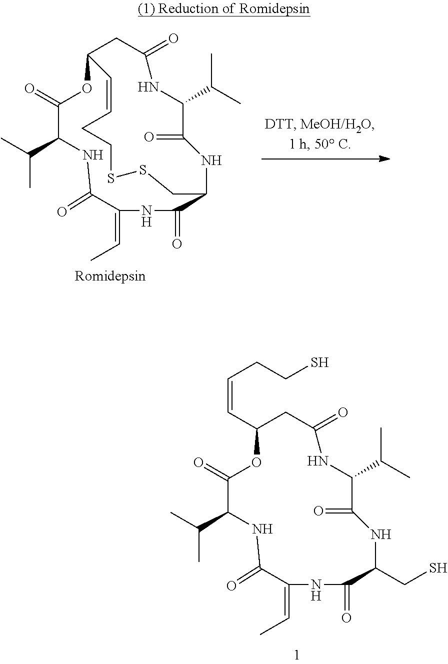 [SMILES: C/C=C1/NC(=O)[C@H]2CSSCC/C=C\[C@H](CC(=O)N[C@H](C(C)C)C(=O)N2)OC(=O)[C@H](C(C)C)NC1=O ; C/C=C1/NC(=O)[C@H](CS)NC(=O)[C@@H](C(C)C)NC(=O)C[C@H](/C=C\CCS)OC(=O)[C@H](C(C)C)NC1=O]

A solution of Romidepsin (90.0 mg, 0.17 mmol) and DTT (26.0 mg, 0.17 mmol) in MeOH/H₂O (1/1, 1.81 mL) was stirred at 50° C. for 1.0 h. The reaction was monitored by HPLC and LC/MS. After 1.0 hour, the reaction mixture was concentrated under reduced pressure to give reduced Romidepsin (1) ESI-MS (m/z): calcd 542.71; obsd 543.52 [M+H]⁺.

### (2) Preparation of SMCC-Romidepsin-Spy

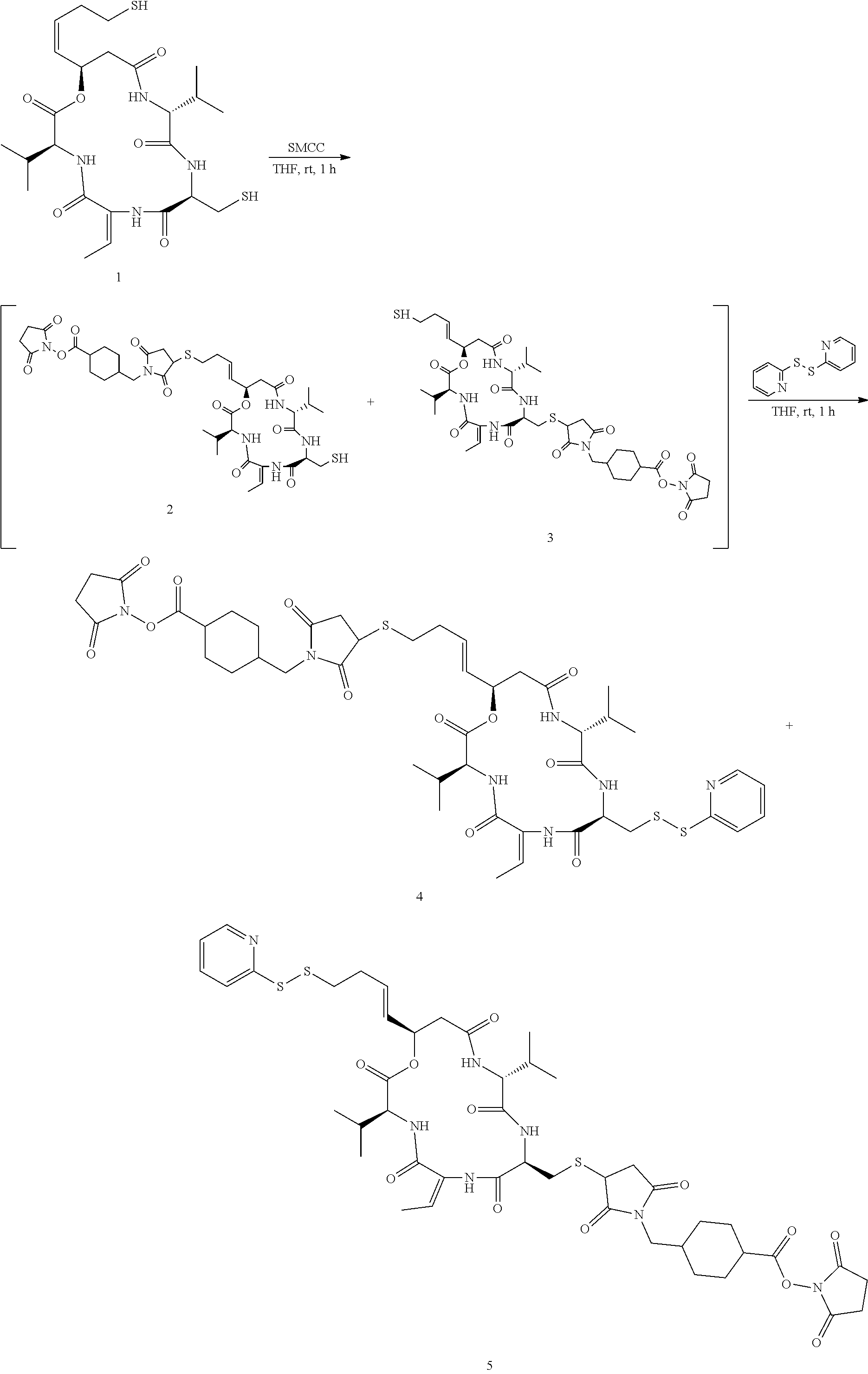

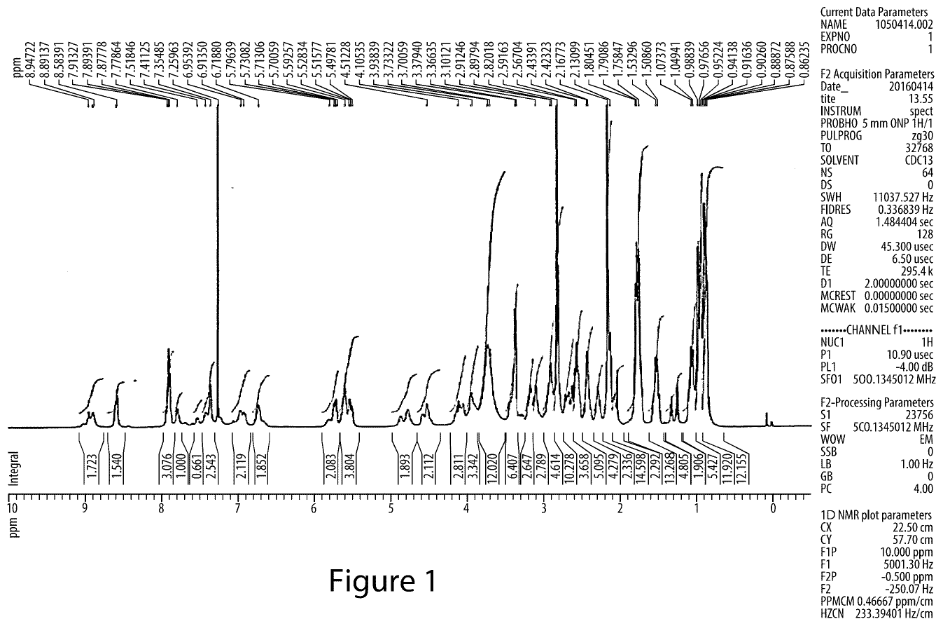

Reduced Romidepsin (1) in THF (1.0 mL) was added SMCC (2,5-dioxopyrrolidin-1-yl 4-((2,5-dioxo-2,5-dihydro-1H-pyrrol-1-yl)methyl)cyclohexane-1-carboxylate, 56.0 mg, 0.17 mmol). The solution was stirred in room temperature for 1 h and monitor the reaction by checking LC/MS. SMCC-Romidepsin mixture (2) and (3) showed ESI-MS (m/z): calcd 877.04; obsd 877.64 [M+H]⁺. Then the reaction mixture was added 1,2-di(pyridin-2-yl)disulfane and stirred for 1.0 h. in room temperature. The reaction was monitored by HPLC and LC/MS. Then the mixture was concentrated under reduced pressure, and purified by C18 silica gel chromatography (0-5% MeOH in dichloromethane) to give a mixture of 2,5-dioxopyrrolidin-1-yl 4-((3-(((E)-4-((3S,9R,12R,16R,E)-6-ethylidene-3,12-diisopropyl-2,5,8,11,14-pentaoxo-9-((pyridin-2-yldisulfanyl)methyl)-1-oxa-4,7,10,13-tetraazacyclohexadecan-16-yl) but-3-en-1-yl)thio)-2,5-dioxopyrrolidin-1-yl)methyl)cyclohexane-1-carboxylate (5) and 2,5-dioxopyrrolidin-1-yl 4-((3-((((3S,9R,12R,16R,E)-6-ethylidene-3,12-diisopropyl-2,5,8,11,14-pentaoxo-16-((E)-4-(pyridin-2-yldisulfanyl) but-1-en-1-yl)-1-oxa-4,7,10,13-tetraazacyclohexadecan-9-yl)methyl)thio)-2,5-dioxopyrrolidin-1-yl)methyl)cyclohexane-1-carboxylate (6) (23.0 mg, 14.0% yield). ESI-MS (m/z): calcd 986.18; obsd 986.67 [M+H]+. NMR was shown as FIG. 1.

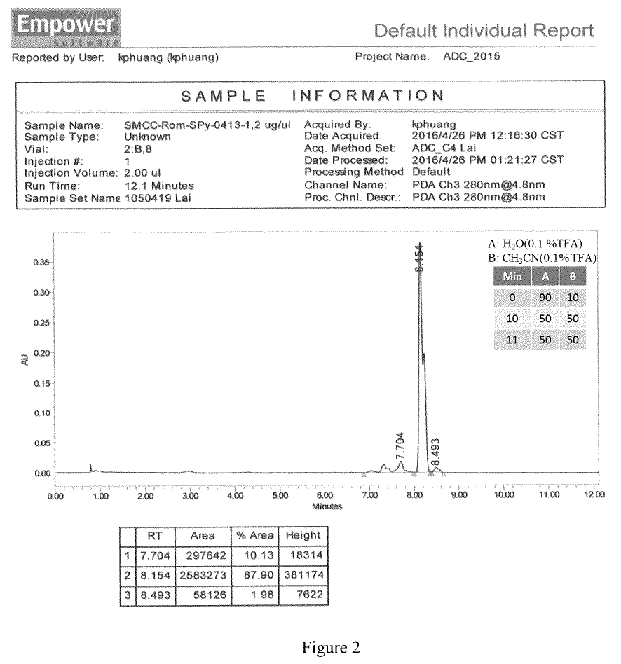

Reverse-phase HPLC analysis was performed on a Waters Acquity UPLC system on a BEH300 C4, 2.1×50 mm column, 1.7 μm particle size. The mobile phase consisted of buffer A (H₂O (0.1% TFA)) and buffer B (CH3CN (0.1% TFA)). A gradient of 10˜50% buffer B was run at 0.2 mL/min over 12 min. The result was shown as FIG. 2.

### 2. Preparation of TMB-355-Biotin:

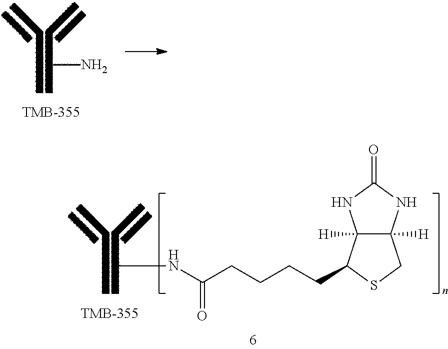

The ADCs with biotin as a payload offers an easy way of testing the conjugation between TMB-355 and small molecules. 8.0 μL 2,5-dioxopyrrolidin-1-yl 5-((3aS,4S,6aR)-2-oxohexahydro-1H-thieno-[3,4-d]imidazol-4-yl) pentanoate (20 mM in DMSO) was added to a solution of Anti-CD4 monoclonal antibody (TMB-355) (180 μL (10.0 mg/mL); in a buffer (containing 50 mM potassium phosphate, 50 mM NaCl, 2 mM EDTA, pH 6.5). The reaction mixture was stirred at 37° C. and stirred for 17 hours. The antibody preparation was desalted and concentrated by using Desalt and concentrate the antibody preparation using the Amicon Ultra-15 centrifugal filter device with 30 kDa NMWL in pH 7.4 Histidine buffer to give TMB-355-Biotin (6). For sterile filtration, the TMB-355-Biotin (6) was filtered by 0.22 μm disposable filter. The final protein concentration of TMB-355-Biotin (6) was determined by calculating molar extinction coefficient of the absorbance of TMB-355-Biotin at 280 nm.

### 3. Preparation TMB-355-SMCC-Romidepsin-Spy:

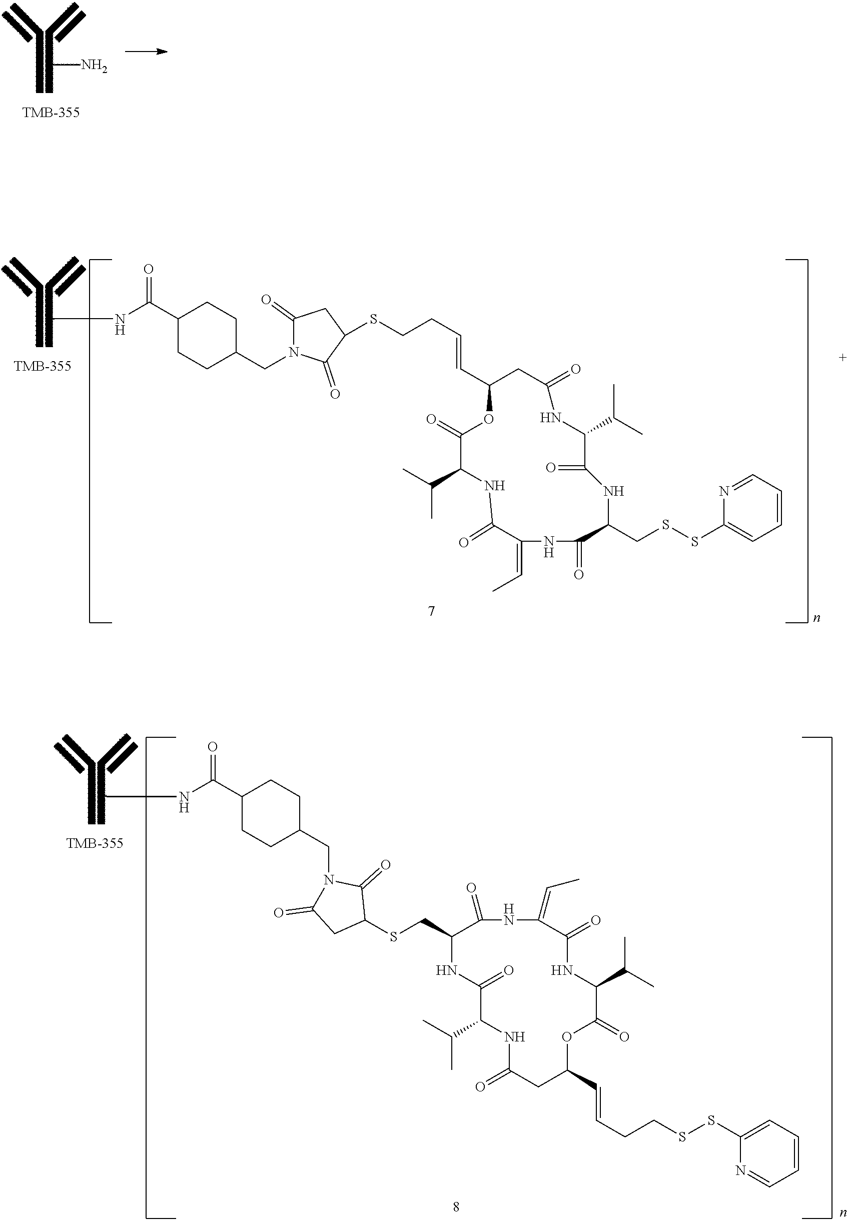

32 μL SMCC-Romidepsin-Spy (10 mM in DMSO) was added to a solution of Anti-CD4 monoclonal antibody (TMB-355) (400 μL (5.0 mg/mL); in a buffer (containing 50 mM potassium phosphate, 50 mM NaCl, 2 mM EDTA, pH 6.5). The reaction mixture was stirred at 37° C. and stirred for 21 hours. The antibody preparation was desalted and concentrated by using Desalt and concentrate the antibody preparation using the Amicon Ultra-15 centrifugal filter device with 30 kDa NMWL in pH 7.4 Histidine buffer to give TMB-355-SMCC-Romidepsin-Spy (7) and (8). For sterile filtration, the TMB-355-SMCC-Romidepsin-Spy (7) and (8) was filtered by 0.22 μm disposable filter. The final protein concentration of TMB-355-SMCC-Romidepsin-Spy (7) and (8) was determined by calculating molar extinction coefficient of the absorbance of TMB-355-SMCC-Romidepsin-Spy at 280 nm.

### 4. Analysis of TMB-355-ADC Products by Reverse-Phase HPLC

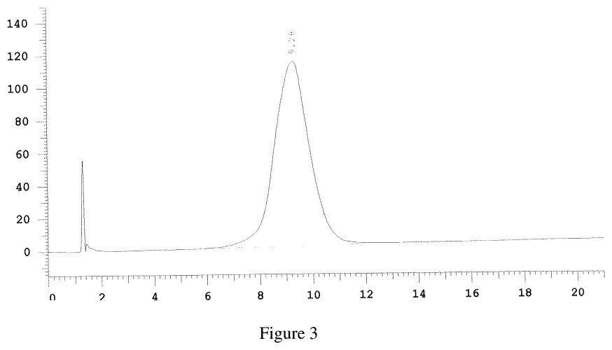

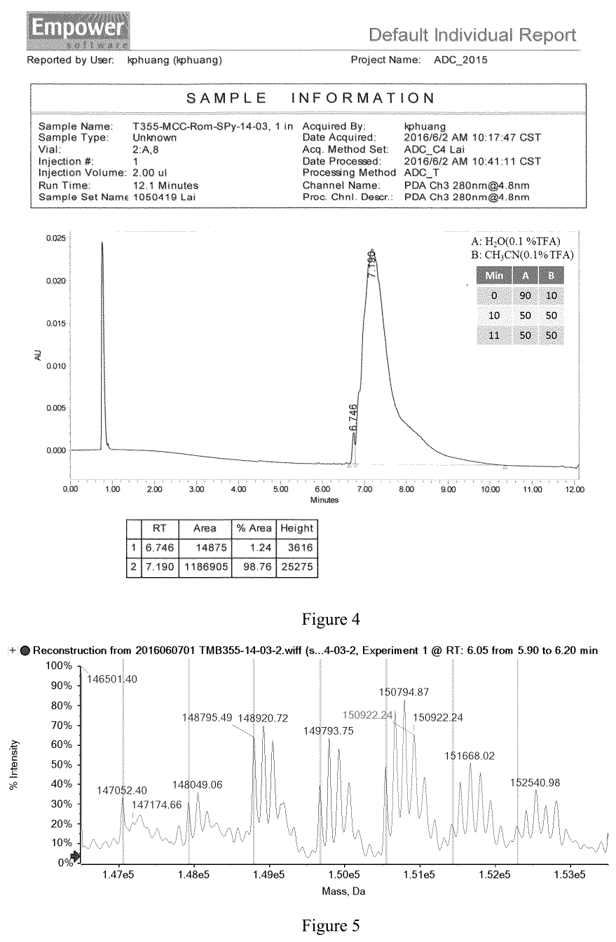

TMB-355-Biotin (6) and TMB-355-SMCC-Romidepsin-Spy (7) and (8) were analyzed by Reverse-phase HPLC by Waters Acquity UPLC system on a BEH300 C4, 2.1×50 mm column, 1.7 μm particle size. The mobile phase consisted of buffer A (H₂O (0.1% TFA)) and buffer B (CH₃CN (0.1% TFA)). A gradient of 10˜50% buffer B was run at 0.2 mL/min over 12 min. The result of TMB-355-Biotin (6) was shown as FIG. 3, and the results of TMB-355-SMCC-Romidepsin-Spy (7) and (8) were shown as FIG. 4.

### 5. Analysis of TMB-355-SMCC-Romidepsin-Spy by LC/MS

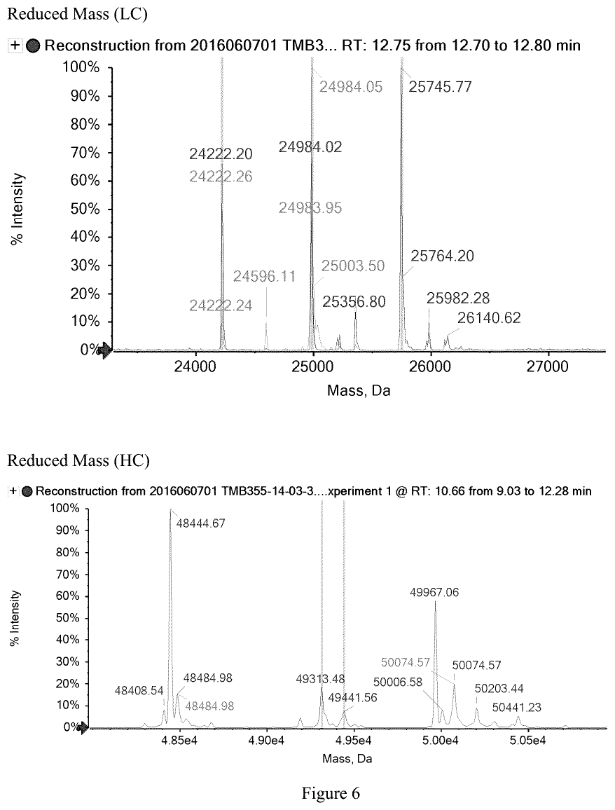

The ADC samples of the intact form and reduced form were analyzed using a TripleTOF™ 5600 coupled with Waters Acquity UPLC system. A Waters ACQUITY UPLC column (Waters, C4 BEH300, 1.7 μm, 1.0 mm×50 mm) was used for separation at the temperature of 80° C. The gradient was generated at a flow rate of 50 μL/min using 0.1% aqueous formic acid (FA) for mobile phase A and ACN/0.1% aqueous FA for mobile phase B. Initially 5% B was held for 3 min and then increased to 20% B in 1 min, to 50% B in additional 4.9 min. 30 μg of sample was deglycosylated for 20 h at 37° C. with PNGase F prior to analysis. The intact and reduced LC/MS results of TMB-355-SMCC-Romidepsin-Spy (7) and (8) were shown as FIG. 5 and FIG. 6.

### 6. Analysis of TMB-355 and ADC Products by Gel Electrophoresis

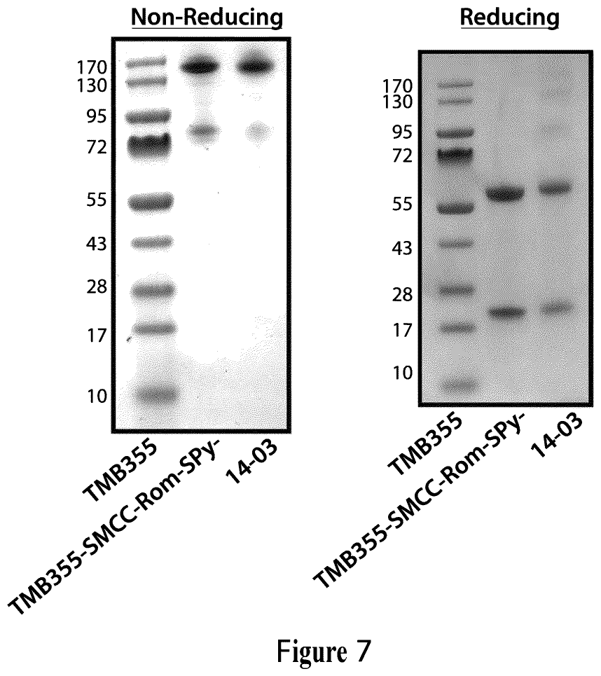

The purified TMB-355 naked antibody, TMB-355-Biotin (6), and TMB-355-SMCC-Romidepsin-Spy (7) and (8) were analyzed on 12% SDS-PAGE under reducing or non-reducing conditions, followed by Coomassie Brilliant Blue staining. For Western blot analysis, TMB-355-SMCC-Romidepsin-Spy (7) and (8) were separated by SDS-PAGE under reducing and non-reducing conditions and then electrophoretically transferred onto polyvinylidine difluoride membranes (Amersham Biosciences). After protein transfer, the membranes were treated with the blocking buffer followed by incubation with HRP-conjugated goat anti-human IgG (H+L). Then, the bands were visualized by 3,3′-diaminobenzidine (Sigma) as a peroxidase substrate. The result was shown in FIG. 7.

### 7. Biotinylation Assay

100 μL of IgG in a coating buffer at a concentration of 1 μg/ml was added to and coated on each well of a plate. The plates were sealed and incubated at 4° C. overnight. The wells were aspirated and washed with 300 μL/well of PBST (0.05% Tween 20) 3 times. The wells were blocked by adding 300 μL/well of PBS-5% skim milk and incubating at 37° C. for 1 hour. The wells were aspirated and washed with 300 μL/well of PBST (0.05% Tween 20) 3 times. 100 μL of 100 ng TMB-355-Biotin (6) diluted with PBS was added to each well and the plates were then incubated at 37° C. for 1 hour. The wells were aspirated and washed with 300 μL/well of PBST (0.05% Tween 20) 3 times. 100 μL/well Streptavidin (1:10000) was added to each well and the plates were then incubated at 37° C. for 1 hour. The wells were aspirated and washed with 300 μL/well of PBST (0.05% Tween 20) 3 times. 100 μL/well of TMB was added to each well and the plates were then incubated at 37° C. for 10 minutes. The color development was stopped by adding 100 μL of IN HCl. And the plates were measured at absorbance of 450-650 nm by using an ELISA reader.

### 8. Binding Affinity

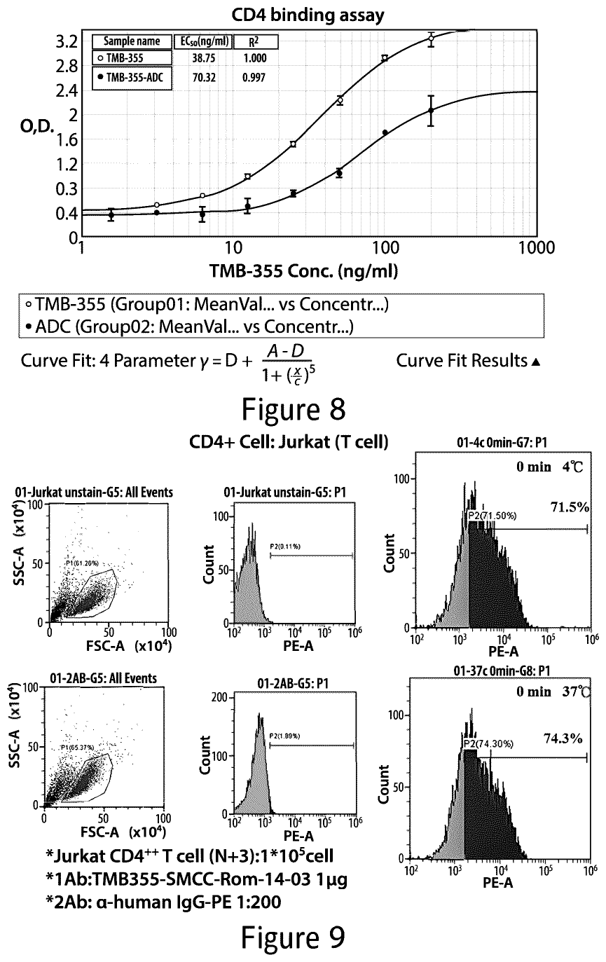

100 μL of anti-human IgG (Fc) in PBS at concentration of 0.5 μg/mL was added to each well of a plate. The plate was sealed and incubated at 25° C. overnight. The wells were blocked by adding 200 μL/well of PBS-1% BSA and incubating at 25° C. for 1 hour. The wells were aspirated and washed with 200 μL/well of TBST (0.05% Tween 20) 3 times. 50 μL of TMB-355-SMCC-Romidepsin-Spy (7) and (8) diluted with PBS at concentration of 200, 100, 50, 25, 12.5, 6.25, 3.125 and 1.56 ng/ml were added to the wells. Then the plate was incubated at 25° C. for 1 hour. The wells were aspirated and washed with 200 μL/well of TBST (0.05% Tween 20) 3 times. 100 μL of biotinylated sCD4 in PBS at concentration of 75 ng/ml was added to each well and the plate was incubated at 25° C. for 1 hour. The wells were aspirated and washed with 200 μL/well of TBST (0.05% Tween 20) 3 times. 100 μL of HRP-conjugated streptavidin (1:10000) was added to each well and the plate was incubated at 25° C. for 30 mins. The wells were aspirated and washed with 200 μL/well of TBST (0.05% Tween 20) 3 times. 100 μL of TMB was added to each well and the plate was incubated at 25° C. for 10 mins. The color development was stopped by adding 100 μL of 0.2 N sulfuric acid. The plate was measured at absorbance of 450 nm using 570 nm as the reference filter by SpectraMax M2e Microplate Reader (Molecular Devices, USA). The data was shown in FIG. 8.

### 9. Cell Culture of CD4 Positive Jurkat T Cell

Cell culture: CD4 Jurkat T cells (human T cell leukemia) were grown in 90% 1640-RPMI (PAN Biotech GmbH, Germany) plus 10% fetal bovine serum (FBS) supplemented with 2 mM L-glutamine (Biochrome, Germany), MEM non-essential amino acids, sodium pyruvate, MEM vitamins, 50 U/mL penicillin/streptomycin, and 50 nM beta-mercaptoethanol (all from Gibco, USA) in a humidified atmosphere at 37° C. and 5% CO₂.

### 10. Enrichment of CD4+ Jurkat T Cells:

For enrichment of CD4 Jurkat T Cells, the Midi-MACS separation system (Miltenyi Biotec, Germany) and CD4 magnetic micro beads (MiltenyiBiote #130-096-533) were used. The Jurkat T cells were harvested and centrifuged at 300×g for 10 mins. Discard the supernatant and resuspend the pellet in 80 μL of buffer per 10⁷ total cells. For depletion of non-targeted CD4⁻ or low CD4⁻ expressing cells, the cell suspension was applied onto the MASC® column and collected flow-through containing unlabeled cells. Then, the column was washed with 3 mL of FACS buffer. Removed column from the separator and place it on a suitable collection tube. Pipette 5 mL of buffer onto the column. Immediately flush out the magnetically labeled non-CD4⁺ T cells by firmly pushing the plunger into the column. Collect unlabeled cells that pass through, representing the enriched CD4⁺ T cells, representing the enriched CD4⁺ T cells. The isolated CD4⁺ Jurkat T cells were then stained with anti CD4⁻FITC antibody (BD Biosciences, USA), and their purity was detected with a Flow cytometry-Accuri (BD Biosciences). The purity of cells after two cycles of MACS sorting was determined to reach above 70%. The result was shown in FIG. 9.

### 11. HDAC Inhibition Assay

Cellular HDAC enzymatic assay was conducted for 3 days with the CD4 enriched Jurkat T lymphocyte cells or B lymphocyte cells treated with various amounts of drugs using FLUOR DE LYS® HDAC fluorometric cellular activity assay kit (Enzo Life Sciences, Inc., cat. No: BML-AK503-0001). The assay was performed following the manufacturer's instructions. Briefly, a serial ten-fold dilution for eight points of drugs (Romidepsin, TMB-355-ADC, and TMB-355) was used. The final concentration in the test was ranging from 1×10³ nM to 1×10⁻⁴ nM for Romidepsin, from 6.67×10² nM to 6.67×10⁻⁵ nM for TMB-355-ADC and TMB-355. The Fluor de Lys substrate was added in a culture media. After 30 min 37° C., 1 volume of the developer was added. After 15 min 37° C., the fluorophore was excited with 355 nm light and the emitted light (450 nm) was being detected on a fluorometric plate reader (Clariostar: BMG, Buckinghamshire, UK). For each experiment, controls (containing CD4 enriched Jurkat T lymphocyte cells or Ramos B lymphocyte cells and buffer), a blank incubation (containing buffer but no CD4 enriched Jurkat T lymphocyte cells or Ramos B lymphocyte cells) and samples (containing compound dissolved in DMSO or ADCs dissolved in PBS and further diluted in medium) were run in parallel. In each test the blank value was subtracted from both the control and the sample values. The control sample represented 100% of substrate deacetylation. For each sample the fluorescence was expressed as a percentage of the mean value of the controls. When appropriate IC₅₀-values (concentration of the drug, needed to reduce the amounts of metabolites to 50% of the control) were computed using GraphPad Prism 6.0 (GraphPad Software Inc., San Diego, Calif.) analysis for graded data. The resulting IC₅₀ value of each drug in Jurkat T lymphocyte cells and Ramos B lymphocyte cells is listed in Table 3.

TABLE 3
The IC₅₀ value of Cellular HDAC enzymatic assay for
Romidepsin, TMB-355-ADC, and TMB-355 in Jurkat T
lymphocyte cells and Ramos B lymphocyte cells

| | HDAC inhibition assay | | |
|---|---|---|---|
| | Romidepsin | TMB355- | TMB- |
| Cell line | (nM) | ADC (nM) | 355(nM) |
| Jurkat CD4⁺ | 1.6 | 4.6 | >666.7 |
| (human T lymphocyte cells) | | | |
| Ramos(human B lymphocyte) | 3.9 | 48.1 | >666.7 |

### 12. Cytotoxicity Assay

In vitro cytotoxicity assay was conducted for 3 days with the CD4 enriched Jurkat T cells and treated with various amounts of drugs using CellTiter-Glo® Luminescent Cell Viability Assay Kit (Promega, G7571). The assay was performed following the manufacturer's instructions. Briefly, cells were plated in 96-well plates at 1,0000 cells/well in 100 μl RPMI 1640 medium with 10% heat-inactivated Fetal Bovine Serum. The following day, a serial ten-fold dilution for eight points of drugs (Romidepsin, TMB-355-ADC, and TMB-355) was used. The final concentration in the test was ranging from 1×10³ nM to 1×10⁻⁴ nM for Romidepsin, from 6.67×10² nM to 6.67×10⁻⁵ nM for TMB-355-ADC and TMB-355. The cells were then incubated at 37° C. for 72 h. Subsequently, 100 μL/well of CellTiter-Glo reagent was added and the cells were shaken for 2 minutes and then incubated for an additional 10 minutes at room temperature. After the incubation time, luminescence (light) was measured in a luminometer (Clariostar: BMG, Buckinghamshire, UK). The IC₅₀ corresponds to the drug concentration which achieves 50% activity of untreated control cultures. IC₅₀ values were calculated using the software package Prism 6.0 (GraphPad Software Inc., San Diego, Calif.) with variable slope option. The resulting IC₅₀ value of each drug in Jurkat T lymphocyte cells and Ramos B lymphocyte cells is listed in Table 4.

TABLE 4
The IC₅₀ value of Luminescent Cell Viability Assay
for Romidepsin, TMB-355-ADC, and TMB-355 in Jurkat
T lymphocyte cells and Ramos B lymphocyte cells

| | CellTiter-Glo Luminescent Cell | | |
|---|---|---|---|
| | Viability Assay | | |
| | Romidepsin | TMB355- | TMB- |
| Cell line | (nM) | ADC (nM) | 355(nM) |
| Jurkat CD4⁺ | 1.6 | 4.1 | >666.7 |
| (human T lymphocyte cells) | | | |
| Ramos(human B lymphocyte) | 2.2 | 49.9 | >666.7 |

### 13. Flow Cytometry-Based Internalization Assay:

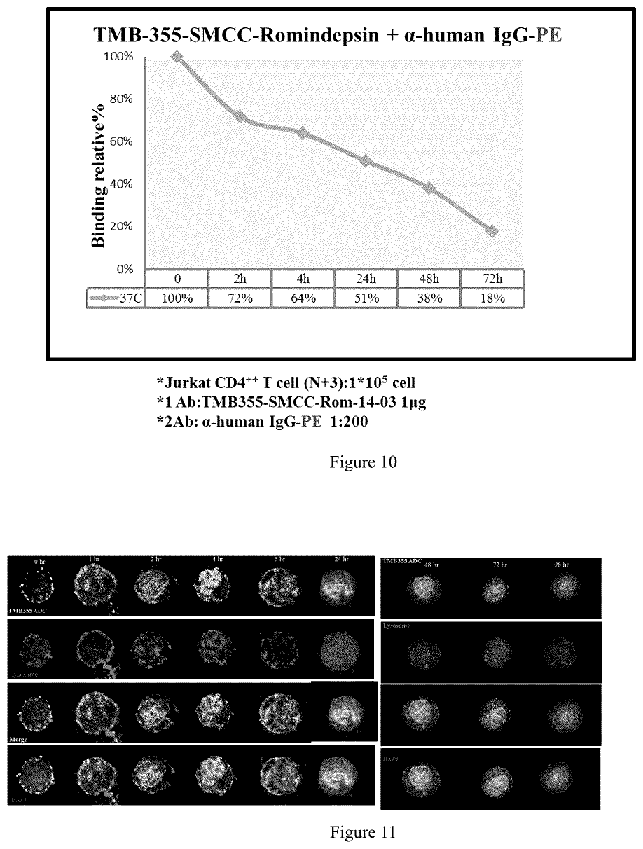

Jurkat T cells were trypsinized and then harvested and resuspended in FAC buffer. Cells were pre-incubated with 5 μg/ml TMB-355-SMCC-Romidepsin-Spy (7) and (8) in FAC buffer on ice for 60 min, washed three times with FAC buffer, and then incubated at 37° C. for time intervals. Next, anti-human IgG PE (1:200) was added to the cells. The cells were incubated at 4° C. for 1 hour and then washed by 2 mL FACS buffer. The supernatant was discarded. The cells were analyzed by flow cytometry and the results are shown in FIG. 10.

### 14. Deconvolution Microscopy-Based Internalization Assay:

Put coverslip in each well of 24-well plate. Added 0.5 mL of coating buffer to each well (cover the whole glass coverslips) for 30 min. Removed coating buffer and air dried the coverslips. Counted cell number of Jurkat T cells and subjected TMB-355-SMCC-Romidepsin-Spy (7) and (8) (Conc.: 1 μg/1*105 cells in 200 μl FACS buffer, total 4 mL), mixed well and incubated on ice for 1 hr. Next, the cells were spun down with 200×g, at 4° C. for 5 min and then discard the supernatant. The Jurkat cells were washed by 10 mL ice-cold FACS buffer three times. Added secondary antibody (αhIgG-FITC, 1:200 diluted by FACS buffer) and anti-lysosome antibody (RFP, 1:50 diluted by FACS buffer), 1*10⁵/100 μl, total 2 mL, mixed well, then incubated at 4° C. for 1 hr. Repeat washing and replaced supernatant for three to five times. Then, seeded 5,000 cells/well by RPMI medium, then incubated at 37° C. or 4° C. for indicated period. At each time point, picked up and fixed the slide in 4% paraformaldehyde at 4° C. for 30 minutes. The slices were mounted with ProLong Gold antifade reagent and left embedded slides in the dark O/N for drying. Cells were imaged on a Deltavision deconvolution microscope to determine TMB-355-SMCC-Romidepsin-Spy (7) and (8), endosome, and lysosome colocalization. The results were shown in FIG. 11.

### 15. TMB-365 ADCs Linker-Inhibitor Intracellular Drug Release Sample Analysis by Using LC-MS/MS.

A sufficient quantity (approximately 7×10⁶ cells) cells were seeded CD4 SUP-T1 cells and CD4⁻ Ramos cells in a 24-well plate, then treated with TMB-365 ADCs, and quantify the intracellular free inhibitors (TMB365 and Cabotegravir) over time using LC/MS-MS. SUP-T1 and Ramos cells were cultured until the total cell count reaches approximately 7×10⁶ cells, then seeded at 1×10⁵ cells per well in a 24-well plate and incubated overnight at 37° C. with 5% CO2. Removed the culture medium at the following day, and 1 mL of medium containing 10 nM TMB-365 ADCs was added to each well, followed by continued incubation. Cells were collected at 0, 3, 6, 24, 48, and 72 hours. The culture medium was removed, 200 μL of Trypsin-EDTA was added to each well and incubated at 37° C. with 5% CO2 for 5 minutes, followed by the addition of 800 μL culture medium to stop the reaction. The contents (˜1 mL) were transferred to 1.7 mL microcentrifuge tubes, and each well was washed with 500 μL of culture medium, combining the wash with the previous contents (˜1.5 mL total). After centrifuging at 800 rcf for 5 minutes and discarding the supernatant, the cell pellet is washed twice with 0.5 mL DPBS and combined from duplicate wells. The cells were resuspended in 0.2-1 mL PBS, and 10 μL of this suspension is mixed with 10 μL trypan blue for counting. An aliquot containing 1×10⁵ cells is centrifuged at 800 rcf for 5 minutes, the supernatant discarded, and the pellet stored at −80° C. For LC/MS-MS analysis, 50 μL of acetonitrile (ACN) was added to the cell pellet and mixed thoroughly. A 30 μL aliquot of this mixture was added to 90 μL of acetonitrile containing 50 ng/mL internal standard, mixed thoroughly, and centrifuged at 10,000 rcf for 10 minutes. The supernatant was collected for LC/MS-MS analysis to quantify the total intracellular free inhibitor content. The mobile phase condition of LC-MS/MS was displayed in Table 5 below.

TABLE 5
Method of LC-MS/MS in example 15

| Total time | Flow rate | | |
|---|---|---|---|
| (min) | (μL/min) | A % | B % |
| 0 | 800 | 90 | 10 |
| 0.5 | 800 | 90 | 10 |
| 1 | 800 | 10 | 90 |
| 3 | 800 | 10 | 90 |
| 4 | 800 | 90 | 10 |
| 4.5 | 800 | 90 | 10 |

Mobile phase: (A) 0.1% FA + H2O + (B) 0.1% FA + Acetonitrile

### Results

We follow the procedure from US2007/129290, to dissolve DTT in MeOH/H₂O (1/1). The disulfide bond of Romidepsin will be reduced to a pair of —SH. The —SH pair will easily form disulfide bond again so that the reaction with free —SH group should be done immediately. Therefore, SMCC was added into the reaction mixture once the disulfide bond of Romidepsin been reduced. The coupling reaction between SMCC and reduced romidepsin in MeOH/H₂O (1/1) did not give acceptable yield, so we monitor the romidepsin reduction rate and pump dried the solution and then add CHCl₃ as solvent to react with SMCC to give acceptable reaction yield.

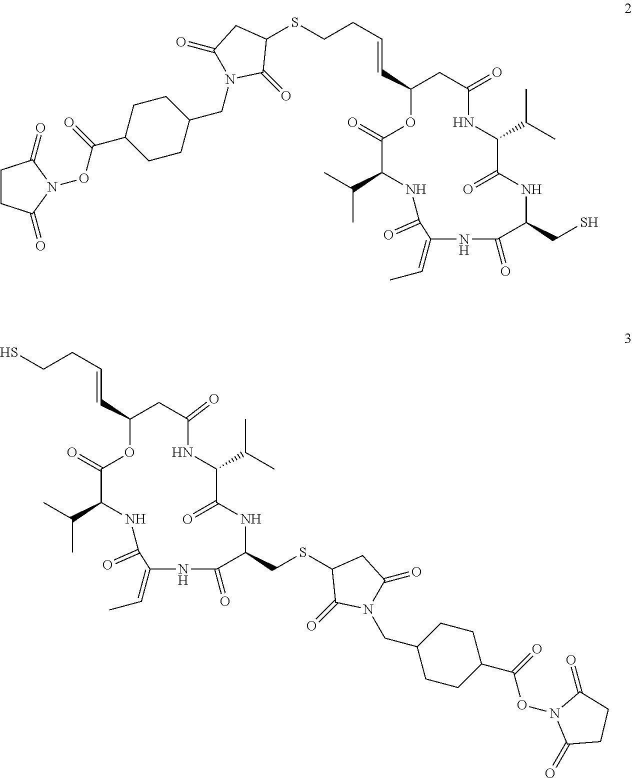 [SMILES: C/C=C1/NC(=O)[C@H](CS)NC(=O)[C@@H](C(C)C)NC(=O)C[C@H](/C=C/CCSC2CC(=O)N(CC3CCC(C(=O)ON4C(=O)CCC4=O)CC3)C2=O)OC(=O)[C@H](C(C)C)NC1=O ; C/C=C1/NC(=O)[C@H](CSC2CC(=O)N(CC3CCC(C(=O)ON4C(=O)CCC4=O)CC3)C2=O)NC(=O)[C@@H](C(C)C)NC(=O)C[C@H](/C=C/CCS)OC(=O)[C@H](C(C)C)NC1=O]

If the reaction further stirred in room temperature, the Mass spectrum will show a peak of MW 761. We assume that the free —SH group reacted with Osu group from SMCC, to form compound A. This result suggested that we better put a protecting group on free —SH group.

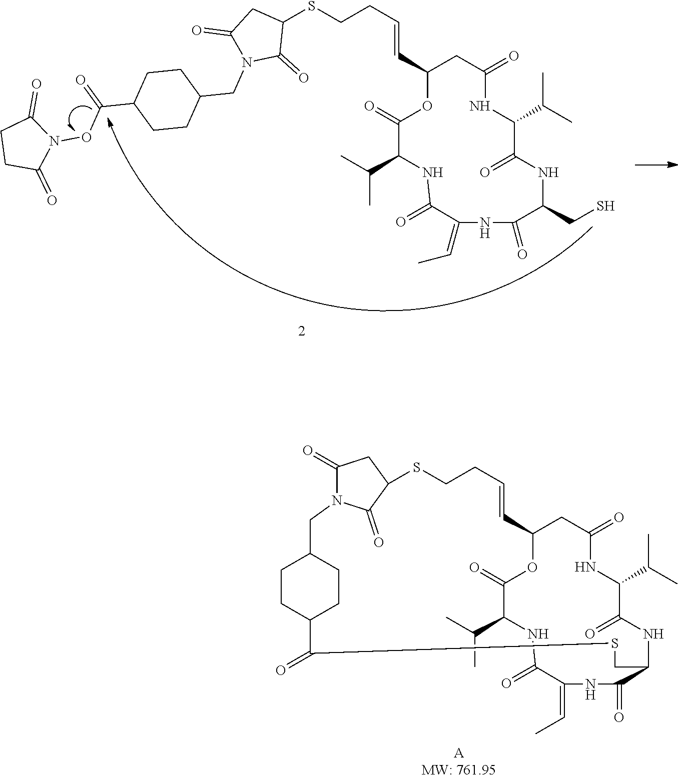 [SMILES: C/C=C1/NC(=O)[C@H](CS)NC(=O)[C@@H](C(C)C)NC(=O)C[C@H](/C=C/CCSC2CC(=O)N(CC3CCC(C(=O)ON4C(=O)CCC4=O)CC3)C2=O)OC(=O)[C@H](C(C)C)NC1=O ; C/C=C1/NC(=O)[C@H]2CSC(=O)C3CCC(CC3)CN3C(=O)CC(SCC/C=C/[C@@H](CC(=O)N[C@H](C(C)C)C(=O)N2)OC(=O)[C@H](C(C)C)NC1=O)C3=O]

To reduce the yield of compound A, we tried to lower the reaction temperature in SMCC and reduced romidepsin reaction. However, we found very low yield of SMCC-romidepsin formed in 0° C. So, we kept the condition under room temperature.

From the literature, we found 1,2-di(pyridin-2-yl)disulfane is easily to form a new disulfide bond from this exchange. Therefore, once compound 2 and/or 3 was formed, the reaction mixture was stirred at room temperature for 30 more minutes, and then 1,2-di(pyridin-2-yl)disulfane was added to the solution as a quencher. The possible produces of 4 and 5 was finally generated.

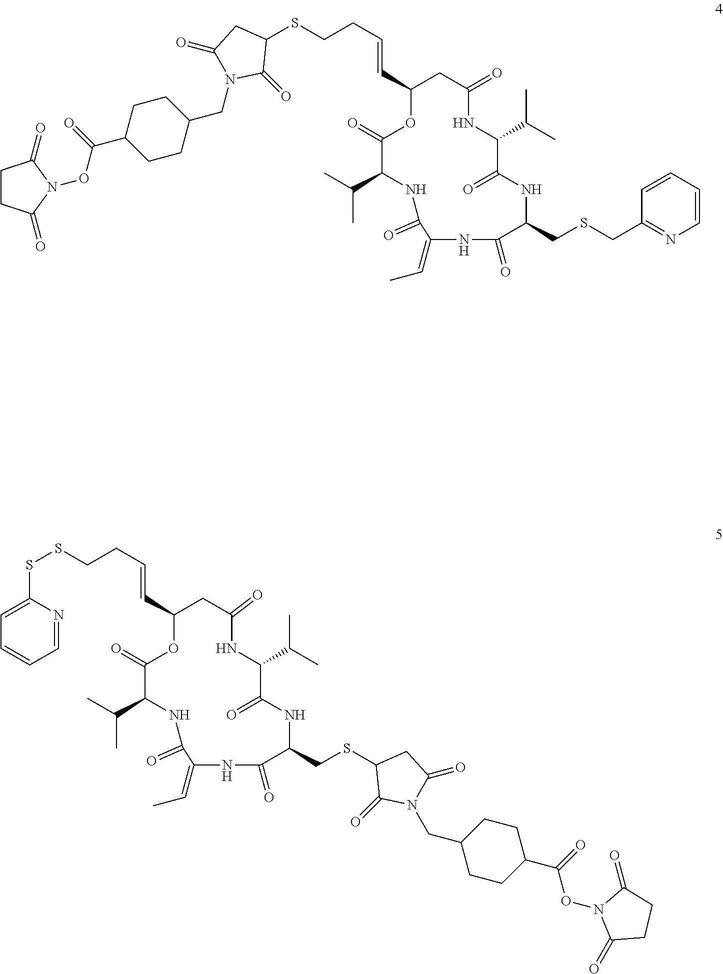 [SMILES: C/C=C1/NC(=O)[C@H](CSCc2ccccn2)NC(=O)[C@@H](C(C)C)NC(=O)C[C@H](/C=C/CCSC2CC(=O)N(CC3CCC(C(=O)ON4C(=O)CCC4=O)CC3)C2=O)OC(=O)[C@H](C(C)C)NC1=O ; C/C=C1/NC(=O)[C@H](CSC2CC(=O)N(CC3CCC(C(=O)ON4C(=O)CCC4=O)CC3)C2=O)NC(=O)[C@@H](C(C)C)NC(=O)C[C@H](/C=C/CCSSc2ccccn2)OC(=O)[C@H](C(C)C)NC1=O]

In another example, TMB-365 based ADC was shown to be taken up by CD4+ SUP-T1 cells, but not by CD4⁻ Ramos cells. The result of the intracellular drug release sample analysis is depicted in Table 6. After 72 hours of ADC treatment, no free inhibitor was detected in CD4⁻ Ramos cells. In contrast, approximately 20% of the original TMB-365 and about 33% of the original Cabotegravir were found intracellularly in CD4⁺ SUP-T1 cells. This result suggests that TMB-365 and Cabotegravir could be successfully internalized by CD4⁺ SUP-T1 cells with ADC treatment.

TABLE 6
Released ratio of intracellular TMB-365 and Cabotegravir.

| | Treatment Compound | | | |
|---|---|---|---|---|
| | 10 nM TMB-365 ADC | | | |
| | Cell line | | | |
| | SUP-T1 (+) | | Ramos (−) | |
| Analysis | TMB-365 | Cabotegravir | TMB-365 | Cabotegravir |
| compound | (%) | (%) | (%) | (%) |
| 0 | BLQ | BLQ | BLQ | BLQ |
| 3 | 19.5 ± 0.1% | 33.3 ± 0.3% | BLQ | BLQ |
| 6 | 19.5 ± 0.1% | 33.4 ± 0.2% | BLQ | BLQ |
| 24 | 19.6 ± 0.2% | 33.4 ± 0.2% | BLQ | BLQ |
| 48 | 19.6 ± 0.2% | 33.3 ± 0.3% | BLQ | BLQ |
| 72 | 19.7 ± 0.2% | 33.6 ± 0.0% | BLQ | BLQ |

BLQ: Below the Limit of Quantification

In conclusion, TMB-355-SMCC-romidepsin-Spy ADC (7) and (8) have been prepared successfully. The HDAC inhibition ability of the TMC-355 ADC showed 10-fold difference between the CD4⁺ Jurkat T cells and the CD4-Ramos B cells. The characterization and evaluation of the conjugate ADC have also been done. The data showed the TMB-355 ADC and TMB-365 ADC could internalize into CD4⁺ cells.

While the present invention has been disclosed by way referred embodiments, it is not intended to limit the present invention. Any person of ordinary skill in the art may, without departing from the spirit and scope of the present invention, shall be allowed to perform modification and embellishment. Therefore, the scope of protection of the present invention shall be governed by which defined by the claims attached subsequently.

What is claimed is:

1. An antibody-drug conjugate (ADC) for treating HIV infection, comprising an antibody that binds to CD4, conjugated via a linker to one or more small-molecule drugs capable of treating or preventing HIV infection.

2. The ADC according to claim 1, wherein the antibody is an anti-CD4, a binding protein, peptide or fragment capable of binding to CD4, or a variant, derivative or modified form thereof.

3. The ADC according to claim 2, wherein the anti-CD4 is TMB-355, TMB-360, TMB-365, TMB-370, or a variant, derivative or modified form thereof.

4. The ADC according to claim 3, wherein the anti-CD4 is TMB-355.

5. The ADC according to claim 3, wherein the anti-CD4 is TMB-365.

6. The ADC according to claim 1, wherein the small-molecule drug is a functional cure-oriented latency reversing agent (LRA) or a treatment-driven antiretroviral (ARV).

7. The ADC according to claim 6, wherein the LRA is a HDAC inhibitor.

8. The ADC according to claim 6, wherein the ARV is an integrase strand transfer inhibitor (INSTI), a reverse transcriptase inhibitor (RTI), a protease inhibitor (PI), a capsid assembly inhibitor (CAI), an entry inhibitor (EI), an attachment inhibitor, or a maturation inhibitor.

9. The ADC according to claim 1, wherein the small-molecule drug is an HDAC inhibitor, which is selected from the group consisting of vorinostat, romidepsin, chidamide, panobinostat, and belinostat.

10. The ADC according to claim 1, wherein the small-molecule drug is Abacavir, Emtricitabine, Lamivudine, Tenofovir disoproxil fumarate, Tenofovir Alafenamide, Zidovudine, Efavirenz, Etravirine, Nevirapine, Atazanavir, Darunavir, Fosamprenavir, Tipranavir, Dolutegravir, Elvitegravir, Bictegravir, Raltegravir, Islatravir, Dapivirine, Rilpivirine, Doravirine, Lenacapavir, or Cabotegravir.

11. The ADC according to claim 1, wherein the linker is selected from the group consisting of
maleimidocaproyl (mc),
maleimidocaproyl-p-aminobenzylcarbamate,
maleimidocaproyl-valine-citrulline-para-aminobenzylcarbamoyl (val-cit),
maleimidocaproyl-peptide-aminobenzylcarbamate linkers,
N-succinimidyl 3-(2-pyridyldithio) proprionate (SPP),
4-succinimidyl-oxycarbonyl-2-methyl-2-(2-pyridyldithio)-toluene (SMPT),
N-succinimidyl 3-(2-pyridyldithio) propionate (SPDP),
N-succinimidyl 4-(2-pyridyldithio) butyrate (SPDB),
2-iminothiolane,
Sacetylsuccinic anhydride,
disulfide benzyl carbamate,
carbonate,
hydrazone linkers,
N-(a-Maleimidoacetoxy) succinimide ester,
N-[4-(p-Azidosalicylamido)butyl]-3′-(2′-pyridyldithio) propionamide (AMAS),
N-[b-Maleimidopropyloxy]succinimide ester (BMPS),
[N-e-Maleimidocaproyloxy]succinimide ester (EMCS),
N-[g-Maleimidobutyryloxy]succinimide ester (GMBS),
Succinimidyl-4-[NMaleimidomethyl]cyclohexane-1-carboxy-[6-amidocaproate] (LC-SMCC),
Succinimidyl 6-(3-[2-pyridyldithio]-propionamido) hexanoate (LC-SPDP),
m-Maleimidobenzoyl-N-hydroxysuccinimide ester (MBS),
N-Succinimidyl [4-26 iodoacetyl]aminobenzoate (SIAB),
Succinimidyl 4-[Nmaleimidomethyl]cyclohexane-1-carboxylate (SMCC),
N-Succinimidyl 3-[2-pyridyldithio]-propionamido (SPDP),
[N-e-Maleimidocaproyloxy]sulfosuccinimide ester (Sulfo-EMCS),
N-[g-Maleimidobutyryloxy]sulfosuccinimide ester (Sulfo-GMBS),
4-Sulfosuccinimidyl-6-methyl-α-(2-pyridyldithio) toluamido]hexanoate) (Sulfo-LC-SMPT),
Sulfosuccinimidyl 6-(3′-[2-pyridyldithio]-propionamido) hexanoate (Sulfo-LC-SPDP),
m-Maleimidobenzoyl-Nhydroxysulfosuccinimide ester (Sulfo-MBS),
N-Sulfosuccinimidyl [4-iodoacetyl]aminobenzoate (Sulfo-SIAB),
Sulfosuccinimidyl 4-[Nmaleimidomethyl]cyclohexane-1-carboxylate (Sulfo-SMCC),
Sulfosuccinimidyl 4-[pmaleimidophenyl]butyrate (Sulfo-SMPB),
ethylene glycol-bis(succinic acid Nhydroxysuccinimide ester) (EGS),
disuccinimidyl tartrate (DST),
1,4,7,10-tetraazacyclododecane-1,4,7,10-tetraacetic acid (DOTA),
diethylenetriaminepentaacetic acid (DTPA), and
thiourea linkers.

12. The ADC according to claim 1, which is a conjugate of an immediate-release anti-CD4, such as TMB-355, TMB-360, TMB-370, and a functional cure-oriented latency reversing agent (LRA), such as an HDAC inhibitor, or a treatment-driven antiretroviral agent (ARV), such as an integrase strand transfer inhibitor (INSTI), a reverse transcriptase inhibitor (RTI), a protease inhibitor (PI), a capsid assembly inhibitor (CAI) or an entry inhibitor (EI).

13. The ADC according to claim 1, which is a conjugate of a sustained-release anti-CD4, such as TMB-365, and a functional cure-oriented latency reversing agent (LRA), such as an HDAC inhibitor, or a treatment-driven antiretroviral agent (ARV), such as an integrase strand transfer inhibitor (INSTI), a reverse transcriptase inhibitor (RTI), a protease inhibitor (PI), a capsid assembly inhibitor (CAI) or an entry inhibitor (EI).

14. A pharmaceutical composition for treating HIV infection comprising a therapeutically effective amount of the ADC set forth in claim 1, and a pharmaceutically acceptable carrier.

15. A method for treating HIV infection in a subject, comprising administering to the subject the ADC set forth in claim 1 in a therapeutically effective amount or the pharmaceutical composition set forth in claim 14.

16. The method according to claim 15, comprising administering to the subject the ADC set forth in claim 1 or the pharmaceutical composition set forth in claim 14, in combination with one or more other therapeutical agents for the treatment of HIV infection.
